(19) United States
(12) Patent Application Publication
(10) Pub. No.: US 2009/0155311 A1
(43) Pub. Date: Jun. 18, 2009

(54) INDOLEAMINE 2,3-DIOXYGENASE PATHWAYS IN THE GENERATION OF REGULATORY T CELLS

(75) Inventors: Wei Chen, Edina, MN (US); Bruce R. Blazar, Golden Valley, MN (US); David Munn, Augusta, GA (US); Andrew Mellor, Augusta, GA (US)

Correspondence Address: MUETING, RAASCH & GEBHARDT, P.A., P.O. BOX 581336, MINNEAPOLIS, MN, 55458-1336, US

(73) Assignee: Med. College of Georgia Research Institute, Inc., Augusta, GA (US)

(21) Appl. No.: 12/158,170

(22) PCT Filed: Jan. 5, 2007
(86) PCT No.: PCT/US07/00404

§ 371 Date: Oct. 20, 2008

Related U.S. Application Data
(60) Provisional application No. 60/756,861, filed on Jan. 7, 2006.

(51) Int. Cl. A61K 39/29; A61K 31/404; A61P 31/12; A61P 37/00

(52) U.S. Cl. 424/227.1; 514/419

(57) ABSTRACT

The present invention provides methods for the control of the generation of regulatory T cells (Tregs) and uses thereof.

### CONTINUING APPLICATION DATA

This application claims the benefit of U.S. Provisional Application Ser. No. 60/756,861, filed Jan. 6, 2006, which is incorporated by reference herein.

### GOVERNMENT FUNDING

The present invention was made with government support under Grant Nos. R01AI34495, 2R37HL56067, R01HL49997, R01HL63453, R01CA103320, R01CA096651, R01CA 112431, HD41187, and AI063402 awarded by the National Institutes of Health. The Government may have certain rights in this invention.

### BACKGROUND

A recently discovered molecular mechanism contributing to peripheral immune tolerance is the immunoregulatory enzyme indoleamine 2,3-dioxygenase (IDO). Cells expressing the tryptophan-catabolizing enzyme IDO are capable of inhibiting T cell proliferation in vitro and reducing T cell immune responses in vivo (U.S. Pat. Nos. 6,451,840 and 6,482,416; Munn et al., Science 1998; 281:1191; Munn et al., J. Exp. Med. 1999; 189:1363; Hwu et al., J. Immunol. 2000; 164:3596; Mellor et al., J. Immunol. 2002; 168:3771; Grohmann et al., J. Immunol. 2001; 167:708; Grohmann et al., J. Immunol. 2001; 166:277; and Alexander et al., Diabetes 2002; 51:356).

IDO degrades the essential amino acid tryptophan (for reviews see Taylor et al., FASEB Journal 1991; 5:2516-2522; Lee et al., Laboratory Investigation, 2003; 83:1457-1466; and Grohmann et al., Trends in Immunology 2003; 24:242-248). Expression of IDO by human monocyte-derived macrophages (Munn et al., J. Exp. Med. 1999; 189:1363-1372), human dendritic cells (Munn et al., Science 2002; 297:1867-1870 and Hwu et al., J. Immunol. 2000; 164:3596-3599), and mouse dendritic cells (Mellor et al., J. Immunol. 2003; 171:1652-1655) allows these different antigen-presenting cells (APCs) to inhibit T cell proliferation in vitro. In vivo, IDO participates in maintaining maternal tolerance toward the antigenically foreign fetus during pregnancy (Munn et al., Science 1998; 281:1191-1193).

IDO has also been implicated in maintaining tolerance to self antigens (Grohmann et al., J. Exp. Med. 2003; 198:153-160), in suppressing T cell responses to MHC-mismatched organ transplants (Miki et al., Transplantation Proceedings 2001; 33:129-130; Swanson, et al. Am J Respir Cell Mol Biol 2004; 30:311-8; Beutelspacher et al. Am J Transplant 2006; 6:1320-30) and in the tolerance-inducing activity of recombinant CTLA4-Ig (Grohmann et al. Nature Immunology 2002; 3:985-1109; Mellor et al. J. Immunol. 2003; 171:1652-1655) and the T cell regulatory functions of interferons (Grohmann et al. J Immunol 2001; 167:708-14; and Baban et al. Int. Immunol 2005; 17:909-919). In these four systems, the immunosuppressive effect of IDO can be blocked by the in vivo administration of an IDO inhibitor, such as 1-methyl-tryptophan (also referred to herein as 1-MT or 1MT).

The transfection of IDO into mouse tumor cell lines confers the ability to suppress T cell responses both in vitro and in vivo (Mellor et al., J. Immunol. 2002; 168:3771-3776). In a Lewis Lung carcinoma model, administration of 1-MT significantly delayed tumor outgrowth (Friberg et al., International Journal of Cancer 2002; 101:151-155). The mouse mastocytoma tumor cell line P815 forms lethal tumors in naive hosts, but is normally rejected by pre-immunized hosts. However, transfection of P815 with IDO prevents its rejection by pre-immunized hosts (Uyttenhove et al., Nature Medicine 2003; 9:1269-1274). Inhibition of tumor growth was entirely dependent on the presence of an intact immune system and was substantially reversed, that is, tumor growth inhibited, by the concomitant administration of 1-MT.

The selective recruitment of IDO⁺ APCs in the tumor-draining (sentinel) lymph nodes of patients with melanoma (Munn et al., Science 2002; 297:1867-1870 and Lee et al., Laboratory Investigation 2003; 83:1457-1466) indicates that tumors take advantage of the immunosuppressive effect of IDO by recruiting a population of IDO-expressing host APCs to present tumor antigens. Similar changes have been seen in breast carcinoma and other tumor-associated lymph nodes. In mouse tumor models the IDO-expressing APCs in tumor-draining lymph nodes are phenotypically similar to a subset of dendritic cells recently shown to mediate profound IDO-dependent immunosuppressive in vivo (Mellor et al., J. Immunol. 2003; 171:1652-1655; and Baban et al. Int. Immunol 2005; 17:909-919). IDO-expressing APCs in tumor-draining lymph nodes thus constitute a potent tolerogenic mechanism.

Plasmacytoid dendritic cells (PDCs) are a unique dendritic cell (DC) subset that plays a critical role in regulating innate and adaptive immune responses (Liu, 2005 Annu Rev Immunol 23:275-306). PDCs sense the microbial pathogen components via Toll-like receptor (TLR) recognition, rapidly produce large amounts of type I interferons (including IFN-α and IFN-β), and activate diverse cell types such as natural killer (NK) cells, macrophages, and CD11c+ DCs to mount immune responses against microbial infections. In addition to stimulating immune responses, increasing evidence suggests that PDCs may also represent a naturally occurring regulatory DC subset (Chen, Curr Opin Organ Transplant 2005; 10:181-185). Under certain circumstances PDCs appear to be able to induce the differentiation of regulatory T cells (Tregs) that downregulate immune responses (Martin et al., Blood 2002; 100:383-390). In humans, PDCs can prime allogeneic naive CD8+ T cells to differentiate into CD8+ suppressor T cells (Gilliet and Liu. J Exp Med 2002; 195:695-704; Wei et al., Cancer Res 2005; 65:5020-5026). It has recently been shown that human PDCs also induce the generation of CD4+ Tregs (Moseman et al., J Immunol 2004; 173:4433-4442). These CD4+ Tregs strongly inhibit autologous or allogeneic T cell proliferation in vitro. Tregs are critical in maintaining self-tolerance and controlling excessive immune reactions (Sakaguchi, Nat Immunol 2005; 6:345-352), so their generation by PDCs is potentially of high biologic significance. However, the mechanism underlying PDC-induced CD4+ Treg generation remains unknown.

### SUMMARY OF THE INVENTION

The present invention includes a method of suppressing the induction of regulatory T cells (Tregs) in a subject, the method including administering to the subject an inhibitor of indoleamine-2,3-dioxygenase (IDO) in an amount effective to suppress the induction of Tregs.

The present invention also includes a method of suppressing the generation or reactivation of regulatory T cells (Tregs) in a subject, the method including administering to the subject an inhibitor of indoleamine-2,3-dioxygenase (IDO) in an amount effective to suppress induction of Tregs.

The present invention also includes a method of reducing immune suppression mediated by regulatory T cells (Tregs) in a subject, the method including administering to the subject an inhibitor of indoleamine-2,3-dioxygenase (IDO) in an amount effective to enhance an immune response.

The present invention also includes a method to reduce the induction of antigen-specific regulatory T cells in a subject, the method including administering to the subject an effective amount of such an antigen in combination with an inhibitor of IDO. In some embodiments, the antigen is a tumor antigen. In some embodiments, the antigen is a viral antigen. In some embodiments, the antigen is an allergen.

The present invention also includes a method to enhance the immune response in a subject to a vaccine antigen, the method including administering to the subject the vaccine antigen, a CpG oligonucleotide (ODN), and an inhibitor of indoleamine-2,3-dioxygenase (IDO).

The present invention also includes a method to enhance the immune response in a subject to a vaccine antigen, the method including administering to the subject the vaccine antigen, a CpG oligonucleotide (ODN), and an inhibitor of GCN2.

The present invention also includes a method to enhance the immune response in a subject to a vaccine antigen, the method including administering to the subject the vaccine antigen and an inhibitor of GCN2.

The present invention also includes a method to induce regulatory T cells in a subject, the method including administering to the subject a metabolic breakdown product of tryptophan, or an analog of a metabolic breakdown product of tryptophan. In some embodiments, the metabolic breakdown product of tryptophan is L-kynurenine, kynurenic acid, anthranilic acid, 3-hydroxyanthranilic acid, quinolinic acid, picolinic acid, analogs thereof, or a combination thereof.

The present invention also includes a method of generating regulatory T cells (Tregs) in a subject, the method including administering to the subject a metabolic breakdown product of tryptophan, or an analog of a metabolic breakdown product of tryptophan.

The present invention also includes a method of increasing immune suppression mediated by regulatory T cells (Tregs) in a subject, the method including administering to the subject a metabolic breakdown product of tryptophan, or an analog of a metabolic breakdown product of tryptophan, in an amount effective to enhance an immune response.

The present invention also includes a method of inducing antigen tolerance in a subject, the method including administering to the subject a metabolic breakdown product of tryptophan, or an analog of a metabolic breakdown product of tryptophan. Some embodiments of the invention include further administering the antigen to the subject.

The present invention also includes a method of inducing a dominant suppressive immune response against an antigen in a subject, the method including administering to the subject a metabolic breakdown product of tryptophan, or an analog of a metabolic breakdown product of tryptophan. In some embodiments, the antigen is the target of an autoimmune response. In some embodiments of the method, the antigen is an alloantigen present in an allograft for transplantation into the subject. Some embodiments include further transplanting the allograft into the subject.

The present invention also includes a method of preventing allograft rejection in a subject, the method including administering to the subject a metabolic breakdown product of tryptophan, or an analog of a metabolic breakdown product of tryptophan, and one or more alloantigens present in the allograft.

The present invention also includes a method of preventing allograft rejection in a recipient, the method including administering a metabolic breakdown product of tryptophan, or an analog of a metabolic breakdown product of tryptophan, to the recipient after the transplantation of the allograft into the recipient.

The present invention also includes a method of preventing graft versus host disease in a recipient, the method including administering to the donor a metabolic breakdown product of tryptophan, or an analog of a metabolic breakdown product of tryptophan, and one or more alloantigens present in the recipient, wherein the metabolic breakdown product of tryptophan, or an analog of a metabolic breakdown product of tryptophan, and the one or more alloantigens present in the recipient are administered to the donor prior to obtaining donor cells from the donor; obtaining donor cells from the donor; and administering the donor cells to the recipient.

The present invention also includes a method of preconditioning a recipient of an allograft to suppress allograft rejection in the recipient, the method including administering to the recipient a metabolic breakdown product of tryptophan, or an analog of a metabolic breakdown product of tryptophan, and one or more alloantigens present in the allograft, wherein the metabolic breakdown product of tryptophan, or an analog of a metabolic breakdown product of tryptophan, and the one or more alloantigens present in the allograft are administered to the recipient prior to allografting; and transplanting the allograft into the recipient.

The present invention also includes a method of generating regulatory T cells (Tregs) in vitro, the method including obtaining naïve CD4+ cells from a subject; obtaining pDCs from the subject; and co-incubating the naïve CD4+ cells and the pDCs with a CpG ODN and a metabolic breakdown product of tryptophan, or an analog of a metabolic breakdown product of tryptophan, for a time sufficient to induce the generation of Tregs.

The present invention also includes a method of suppressing immune mediated allograft rejection in a recipient, the method including obtaining naïve CD4+ cells from the allograft donor; obtaining pDCs from the recipient; and co-incubating the naïve CD4+ cells and the pDCs with a CpG ODN and a metabolic breakdown product of tryptophan, or an analog of a metabolic breakdown product of tryptophan, for a time sufficient to induce the generation of Tregs; administering the induced Tregs to the recipient before, during, and/or after the allograft transplant.

The present invention also includes a method of suppressing immune mediated allograft rejection in a recipient, the method including obtaining naïve CD4+ cells from the allograft donor; obtaining pDCs from the donor; and co-incubating the naïve CD4+ cells and the pDCs with a CpG ODN and a metabolic breakdown product of tryptophan, or an analog of a metabolic breakdown product of tryptophan, for a time sufficient to induce the generation of Tregs; administering the induced Tregs to the recipient before, during, and/or after the allograft transplant.

Also included in the present invention is an isolated cell population preconditioned to minimize graft versus host disease when transplanted into a recipient, the cell population obtained by a method including administering to the donor a metabolic breakdown product of tryptophan, or an analog of a metabolic breakdown product of tryptophan, and one or more alloantigens present in the recipient, wherein the metabolic breakdown product of tryptophan, or an analog of a metabolic breakdown product of tryptophan, and the one or more alloantigens present in the recipient are administered to the donor prior to obtaining donor cells from the donor; and obtaining donor cells from the donor.

The present invention also includes a composition to induce tolerance to an antigen, the composition including a metabolic breakdown product of tryptophan, or an analog of a metabolic breakdown product of tryptophan.

The present invention also includes a composition to induce the generation of regulatory T cells (Tregs), the composition including a metabolic breakdown product of tryptophan, or an analog of a metabolic breakdown product of tryptophan.

The present invention also includes a vaccine for use in immunization protocols for the induction of immune tolerance to an antigen, the vaccine including a metabolic breakdown product of tryptophan, or an analog of a metabolic breakdown product of tryptophan, and the antigen.

The present invention also includes a method to enhance an immune response in a subject including the administration of an effective amount of an inhibitor of a GCN2 kinase. In some embodiments, the method further includes the administration of a vaccine.

The present invention also includes a method to prevent immune suppression mediated by Tregs, the method including the administration of an effective amount of an inhibitor of a GCN2 kinase. In some embodiments, the method further includes the administration of a vaccine.

The present invention also includes a method to enhance an immune response in a subject, the method including administering two or more agents, each agent selected from the group consisting of an inhibitor of indoleamine-2,3-dioxygenase (IDO), a CpG oligonucleotide (ODN), an inhibitor of a GCN2 kinase, a vaccine, and a chemotherapeutic agent.

The present invention also includes a method to prevent immune suppression mediated by Tregs, the method including the administration administering two or more agents, each agent selected from the group consisting of an inhibitor of indoleamine-2,3-dioxygenase (IDO), an inhibitor of a GCN2 kinase, a vaccine, and a chemotherapeutic agent.

In some embodiments of the methods and compositions of the present invention, the inhibitor of IDO is 1-methyl-tryptophan (1-MT). In some embodiments, 1MT may be a D isomer of 1MT, a L isomer of 1MT, or a racemic mixture of 1-MT.

Unless otherwise specified, “a,” “an,” “the,” and “at least one” are used interchangeably and mean one or more than one.

### BRIEF DESCRIPTION OF THE FIGURES

FIGS. 1A-1D show PDC-induced CD4+ Treg generation is antigen and CD28 signaling dependent. In FIG. 1A surface expression of CD80, CD86, HLA-DR on PDCs before or after CpG ODN stimulation for 48 hours was assessed by staining with specific fluorescent Abs (filled) or isotype control Ab (unfilled) and determined by flow cytometry. MFI is indicated. In FIG. 1B CD4+CD25+Foxp3+ Tregs generated in PDC-naive CD4+ T cell priming cultures with or without CpG ODN were determined at day seven. The data presented are aggregate results from five experiments from individual donors and are expressed as the mean±SD. *, p<0.01 (compared CpG ODN vs. no CpG ODN cultures). **, p<0.01 (compared ODN 2216 vs. ODN 2006 cultures). In FIG. 1C anti-CD80/CD86, HLA-DR, and control IgG Abs (10 μg/ml) were added to PDC naive CD4+ T cell priming cultures in the presence of CpG ODN. The percentages of CD4+CD25+Foxp3+ Tregs generated in cultures were determined at day seven. In FIG. 1D anti-CD80/CD86 or HLA-DR Abs (0.1-10 μg/ml), and control IgG Ab (10 μg/ml) were added to PDCs and naive CD4+ T cell priming cultures. The percentage and number of CD4+CD25+Foxp3+ Tregs generated in cultures were determined at day seven. The data in FIG. 1C and FIG. 1D are representative results from one of three reproducible experiments.

FIGS. 2A-2D show that expression of IDO in PDCs plays an important role in CD4+ Treg generation. In FIG. 2A the expression of IDO and loading control β-actin proteins in fresh or cultured PDCs and B cells with or without CpG ODN±1MT for 48 hours were determined by Western blot. Data shown are representative results of two individual donors. FIG. 2B shows surface expression of CD80, CD86, HLADR on PDCs cultured with or without CpG ODN±1MT for 48 hours. The data shown is from one representative experiment with indicated MFIs. In FIG. 2C the percentages of CD4+CD25+Foxp3+ Tregs generated in CpG ODN-PDC and naïve CD4+ T cell priming cultures with or without 1MT were determined at day seven. The data shown is aggregated results from three experiments of different donors and are expressed as the mean±SD. *, p<0.01 (compared 1MT vs. no 1MT cultures). In FIG. 2D CpG ODN-PDC primed CD4+ T cells were plated into an MLR assay where freshly isolated autologous naïve CD4+ T cells were stimulated with irradiated allogeneic PBMC.

FIGS. 3A-3C show blocking IDO activity with 1MT abrogates the generation of functional suppressor activity and hyporesponsiveness of PDC-primed CD4+ T cells. In FIG. 3A CD4+ T cells primed by ODN 2216-PDCs or ODN 2006-PDCs (donor A vs. C) with or without 1MT were plated at graded doses as responders to irradiated PBMC from donor C in an MLR assay. In FIG. 3B ODN 2216-PDC primed CD4+ T cells with or without 1MT (donor A vs. C) were added at graded doses into MLR assays where freshly purified autologous (donor A) or allogeneic (donor B) naïve CD4+ T cells were stimulated with irradiated allogeneic PBMC from donor C or donor D, respectively. In FIG. 3C (left panel), CD4+ T cells primed with CpG ODN-treated B cells, with or without 1MT present during the priming MLR (donor A vs. C) were used as responders in a secondary MLR, using irradiated PBMC from donor C as stimulators. In FIG. 3C (right panel) CD4+ T cells primed with CpG ODN-treated B cells with or without 1MT (donor A vs. C) were plated at graded doses into an MLR assay where freshly purified autologous naïve CD4+ T cells (donor A) were stimulated with irradiated allogeneic PBMC from donor C.

FIGS. 4A-4E show tryptophan metabolites of IDO pathway are critical for CD4+ Treg induction. FIG. 4A is a schematic representation of IDO pathway and Trp catabolism. In FIG. 4B the percentage of CD4+CD25+Foxp3+ Tregs generated in ODN 2216-PDC primed allogeneic naïve CD4+ T cell cultures with or without 1MT and/or KYN was determined at day seven. The data shown are representative results from one of three experiments of different donors. In FIG. 4C naïve CD4+ T cells primed with ODN 2216-PDCs with or without 1MT and/or KYN were plated into MLR assays where freshly isolated autologous naïve CD4+ T cells were stimulated with irradiated allogeneic PBMC. *, p<0.01 (compared to the proliferation of ODN 2216-PDC/1MT primed T cells). In FIG. 4D naïve CD4+ T cells primed with ODN 2216-PDCs with or without 1MT and/or KYN were plated at graded doses as responders to irradiated PBMC from the PDC donor in an MLR assay. FIG. 4E is a schematic representation of KYN-pathway metabolites as a critical signaling event employed by PDC to promote CD4+ Treg generation.

FIGS. 5A-5C show TLR9 ligation enhances Treg suppressor functions. CBA mice were treated with CpG (open symbols) or non-CpG (closed symbols). In FIG. 5A, after 24 hours, Tregs (FIG. 5B) and CD4+CD25− (FIG. 5C) T cells were sorted, and added to cultures containing BM3 T cells and APCs. IDO inhibitor, 1mT, was added to parallel cultures (Δ). Thymidine incorporation was assessed after 72 hours. Data is representative of three separate experiments.

FIGS. 6A and 6B show TLR9-mediated activation of Treg suppressor functions is IDO-dependent. CBA mice were treated with CpG or non-CpG as indicated. After 24 hours, Tregs and CD4+CD25− T cells were sorted from treated mice and added to cultures containing BM3 responder T cells and H-2K^{b}+ stimulator APCs from CBK transgenic mice. BM3 T cell proliferation was measured by thymidine incorporation at 72 hours. In FIG. 6A IDO-WT or IDO-KO were used as Treg sources. In FIG. 6B IDO-WT mice treated with IDO inhibitor (1mT) or vehicle alone were used as Treg sources. Gray bars show BM3 T cell responses in the absence of any sorted CD4+ T cells. Percentages (dotted arrows) show suppression mediated by Tregs from IDO-WT mice relative to Tregs from IDO-KO mice or IDO-WT mice exposed to 1mT, respectively, following CpG treatment. Dotted arrows indicate percent suppression attributable to IDO-induced Treg activation. Data is representative of three separate experiments.

FIGS. 7A-7C show that IDO-activated Tregs suppress allospecific T cell responses in vivo. In FIG. 7A sorted Tregs from CpG or non-CpG treated CBK donor mice were mixed with BM3 T cells and co-injected into CBK recipients. In FIG. 7B, after 96 hours, splenocytes from recipient mice were stained with anti-CD4, anti-CD8, anti-Ti98 (BM3 clonotypic), anti-H-2K^{b} mAbs and analyzed by flow cytometry to detect donor (Ti98+, H-2K^{b}−) BM3 T cells. Graphs report mean number of donor T cells present in spleen of 2-3 recipient mice per group. In FIG. 7C splenic tissues from mice that received resting or activated Tregs were stained with anti-CD8α mAb, which stains BM3 T cells selectively. Data is representative of three separate experiments.

FIGS. 8A-8C show that IDO induces selective CHOP expression in Tregs and enhances the number of FoxP3+ Tregs. In FIG. 8A spleen cells from mice treated with PBS, non-CpG, or CpG were stained for intracellular CHOP, CD4 and CD25 after 24 hours. The left panel shows CHOP and CD4 staining for splenocytes from mice treated with CpG. Percentage gives the fraction of CHOP⁺ cells in total spleen, all of which are CD4⁺. The three right panels show CHOP and CD25 staining profiles for gated CD4+ splenocytes. Percentages give the fraction of CHOP+ Tregs in each treatment group. In FIG. 8B percentages give the fraction of CHOP+ Tregs in wild-type, IDO-KO or GCN2-KO mice treated with PBS or CpG, as shown. FIG. 8C shows FoxP3 and CD25 staining profiles for gated CD4+ splenocytes from wild-type (IDO-WT) or IDO-KO mice treated with CpG or untreated. Percentages give the fraction of FoxP3+CD25+ cells in the total CD4+ population. Data is representative of at least three separate experiments in each case.

FIGS. 9A-9C show suppression of bystander T cells by IDO-activated Tregs. Bystander assays were set up as shown in FIG. 9A, comprising IDO⁺ DCs from TDLNs (CD11c⁺B220⁺); CD8⁺ OT-I T cells (specific for a peptide from chicken ovalbumin); CD4⁺CD25⁺ Tregs from normal B6 spleen; CD4⁺ A1 T cells (specific for a peptide from H-Y); CD11c⁺B220^{NEG} DCs from CBA mice; and a feeder layer of T-depleted spleen cells. The ratio of Tregs to bystander cells was 1:20 (5×10³ Tregs to 1×10⁵ A1 cells). Assays were set up with (FIG. 9C) or without Tregs (FIG. 9B), and with or without 1MT; all assays received cognate peptides for OT-I and A1 cells. OT-I and A1 cells were labeled with CFSE dye; each pair of histograms shows the gated OT-I and A1 populations from a single culture.

FIGS. 10A-10D show suppression of bystander T cells by IDO-activated Tregs. Bystander assays were set up as in FIG. 9, except using thymidine-incorporation to quantitate the combined proliferation of T cells. FIG. 10A shows titration of Tregs added to bystander-suppression assays, in the presence or absence of 1MT, and with or without anti-CD3 (αCD3). FIG. 10B shows pre-activated Tregs (sorted, then cultured for 2 days with αCD3 mitogen, T-depleted spleen cells and IL-2) then added to allo-MLR reactions comprising BM3 T cells (anti-H2K^{b}) plus irradiated B6 spleen cells. The x-axis reflects the nominal number of Tregs initially added to the pre-activation cultures. FIG. 10C shows suppression in bystander assays was not mediated by the CD25^{NEG} (non-Treg) fraction of CD4⁺ cells, but required the addition of sorted CD4⁺CD25⁺ Tregs. These Tregs were typically 90% Foxp3⁺ by intracellular FACS staining (shown in the histogram, day 0), and remained so after IDO-induced activation (day 3). Filled histogram shows isotype control. For Foxp3 staining, cultures were performed without added bystander cells, and the Tregs identified by CD4 expression. FIG. 10D shows bystander assays were set up using IDO-deficient TDLN pDCs (from tumors grown in IDO-KO mice, B6 background), or IDO-KO bystander DCs (CBA background). All Tregs were from normal B6 mice. Arrows show suppression.

FIGS. 11A-11E show that IDO-induced Treg activation requires GCN2-kinase. FIGS. 11A and 11B show GCN2-mediated CHOP induction by IDO. Assays were set up with TDLN pDCs, OT-I cells, Tregs, and feeder layer, but without bystander cells. Antigen for OT-I was added as indicated, and intracellular CHOP expression was measured after 48 hours by flow cytometry. Tregs were followed by CD4 expression. Percentages show the fraction of Tregs that were CHOP⁺. In FIG. 11B, assays were performed using Tregs from either wild-type or GCN2-KO mice (with OVA, without 1MT). FIG. 11C shows functional bystander-suppression assays, comparing Tregs from GCN2-KO or wild-type mice. IDO-induced, Treg-mediated suppression (arrow) was absent in GCN2-KO Tregs. FIG. 11D shows a titration of WT and GCN2-KO Tregs in bystander-suppression assays. In FIG. 11E, Tregs from GCN2-KO or WT hosts were sorted and pre-activated for 2 days with αCD3+IL-2 and assayed for suppressor activity in allo-MLR (BM3 responder T cells).

FIGS. 12A and 12B show that CHOP-KO Tregs are defective in both IDO-induced and αCD3-induced suppressor activity. In FIG. 12A bystander-suppression assays were performed using either CHOP-KO Tregs or WT Tregs, added to assays with CFSE-labeled OT1 and A1 cells. In FIG. 12B Tregs from CHOP-KO or WT hosts were pre-activated for two days with αCD3+IL-2 and assayed for suppressor activity in allo-MLR (BM3 responder T cells).

FIGS. 13A-13D show that Treg activation requires interaction with MHC on the IDO⁺ DCs. FIG. 13A shows FACS assays for CHOP. The first dot-plot shows assays in which Tregs were MHC-matched to the IDO⁺ DCs (both B6 background); the second shows MHC-mismatched Tregs (CBA Tregs, B6 DCs); the third dot-plot shows MHC-matched Tregs but with blocking antibody to IA^{b} (the MHC-II allele expressed by B6 mice). Controls without blocking antibody, or with irrelevant antibody, were similar to the first plot. In FIG. 13B bystander-suppression assays were set up with or without blocking antibody against the MHC-II on the IDO⁺ DCs (IA^{b}). Results by both thymidine incorporation (left) and CFSE (right) are shown. FIG. 13C is a summary of bystander-suppression assays using different haplotype combinations. (+) denote >90% suppression by thymidine incorporation, (−) denotes no suppression compared to 1MT control. FIG. 13D shows bystander-suppression assays using IDO⁺ DCs from TDLNs of tumors grown in either H2-DM^{-/-} (DM-KO) mice, or WT controls.

FIGS. 14A-14C show that IDO-activated Tregs suppress target cells by mechanism that does not require cell-cell contact. FIG. 14A shows bystander-suppression experiments containing the cell populations shown in FIG. 9, performed in transwell chambers with the cells distributed as shown in the diagrams. Feeder cells could be placed in either chamber with identical results; in the studies shown they were in the lower chamber. Bar graphs show proliferation in each chamber, with and without 1MT. In FIG. 14B bystander-suppression assays were performed as in FIG. 10 (not in transwells), with added 1MT, 10× tryptophan (250 uM), or 1MT+25 uM kynurenine. FIG. 14C shows bystander-suppression assays (not in transwells) comparing neutralizing antibodies to IL-10, TGFβ or both together. Control irrelevant antibodies had no effect on suppression.

FIGS. 15A and 15B show IDO-induced Treg activation in vivo. In FIG. 15A recipient mice were pre-loaded with OT-I cells. TDLN DCs (sorted CD11c⁺ cells) were loaded with SIINFEKL (SEQ ID NO:1) peptide and injected subcutaneously into recipients. One group received implantable sustained-release 1MT pellets to block IDO (“IDO blocked”), while the other received control pellets (“IDO active”). After four days, the LNs draining the site of DC injection were removed and the Tregs sorted and tested in vitro for suppressor activity in readout assays comprising A1 cells+CBA DCs+H-Y peptide. FIG. 15B shows adoptive-transfer experiments, as in the previous panel, using either WT or GCN2-KO host mice pre-loaded with CFSE-labeled OT-I cells on the WT or GCN2-KO background (OT-I^{WT} or OT-I^{GCN2-KO}). All mice received TDLN DCs pulsed with SIINFEKL peptide. After four days, lymph nodes draining the DC injection site were analyzed for CFSE cell division and the 1B11 activation antigen, gated on the CD8⁺ CFSE⁺ population. Vertical bars on the top histogram show the 2 SD cutoff for the negative controls for each channel.

FIG. 16 shows antigen presentation to OT-I cells is required to trigger functional IDO enzyme activity.

FIG. 17 shows that αCD3-induced Treg suppressor activity requires cell-cell contact, and is distinct from IDO-induced suppressor activity.

FIG. 18 shows OT-I cells that lack GCN2 are refractory to direct IDO-mediated suppression, but are sensitive to Treg-mediated suppression.

### DETAILED DESCRIPTION OF ILLUSTRATIVE EMBODIMENTS OF THE INVENTION

The present invention demonstrates the role of indoleamine 2,3 dioxygenase (IDO) expression in the induction of regulatory T cells (Tregs), showing that IDO expression is necessary for the induction of CD4+ Tregs by plasmacytoid dendritic cells (also referred to herein as “PDCs” or “pDCs”). The present invention shows that inhibitors of IDO suppress the induction and/or activation of Tregs. A suppression of Tregs is associated with an active immune response. The present invention shows that IDO expression induces of Tregs. The induction of Tregs is associated with the induction of immune tolerance and the suppression of an immune response. The present invention also shows that the induction of Tregs by IDO can be pharmacologically reproduced by the addition of a downstream tryptophan metabolite, including, but not limited to kynurenin (also referred to herein as “KYN” or “kyn”). The observations of the present invention have wide applicability, including for example, in methods for the treatment of autoimmunity, allergic responses, transplant situations, vaccination, and cancer therapy. As used herein, the “induction of Tregs” includes both the generation of Tregs from naïve T cells and the reactivation of quiescent Tregs.

Although most auto-reactive T lymphocytes are regulated and eliminated during thymic development, healthy individuals continue to carry self-reactive cells. T regulatory cells (Tregs) are an immunoregulatory cell type used to control autoimmunity in the periphery. Tregs are CD4 positive. The constitutive expression of CD25 is considered to be a characteristic feature of human Tregs. Thus, Tregs are often CD4+CD25+ T cells.

Tregs are potent suppressors of T cell mediated immunity in a range of inflammatory conditions, including infectious disease, autoimmunity, pregnancy and tumors (Sakaguchi, S, Nat Immunol 2005; 6:345-352). Mice lacking Tregs die rapidly of uncontrolled autoimmune disorders (Khattri et al. Nat Immunol 2003; 4:337-342). In vivo, a small percentage of Tregs can control large numbers of activated effector T cells. Although freshly isolated Tregs exhibit minimal constitutive suppressor functions, ligating the T cell antigen receptor (TCR) in vitro (Thornton et al. Eur J Immunol 2004; 34:366-376), or pre-immunizing mice with high-dose self-antigen in vivo stimulates Treg suppressor functions (Nishikawa et al. J Exp Med 2005; 201:681-686). This requirement for TCR signaling to enhance Treg suppressor functions is paradoxical because most Tregs are thought to recognize constitutively expressed self-antigens (Nishikawa et al. J Exp Med 2005; 201:681-686; Hsieh et al. Immunity 2004; 21:267-277; Fisson et al. J Exp Med 2003; 198:737-746; Kronenberg et al. Nature 2005; 435:598-604). The present invention shows that increased IDO activity stimulates a rapid increase in suppressive functions mediated by splenic Tregs and that the inhibition of IDO activity abrogates suppressive functions.

Tregs of the present invention may express CD4 (CD4⁺) and/or CD25 (CD25⁺). Tregs of the present invention may also be positive for the transcriptional repression factor forkkhead box P3 (FoxP3). Tregs of the present invention may express a high affinity IL-2 receptor. Tregs of the present invention may be CD8⁺ Tregs. Tregs have been studied for more than thirty years and are further reviewed in, for example, Beyer and, Schultze, Blood, 2006; 108(3):804-11; Elkord, Inflamm Allergy Drug Targets, 2006; 5(4):211-7; Ghiringhelli et al., Immunol Rev, 2006; 214:229-38; Jiang et al., Hum Immunol, 2006; 67(10):765-76; Kabelitz et al., Crit Rev Immunol, 2006; 26(4):291-306; Le and Chao, Bone Marrow Transplant, 2007; 39(1):1-9; Sakaguchi et al., Immunol Rev, 2006; 212:8-27; Shevach et al., Immunol Rev, 2006; 212:60-73; Stein-Streilein and Taylor, “An eye's view of T regulatory cells,” J Leukoc Biol, Dec. 28, 2006 (epub ahead of print); and Wing and Sakaguchi, Curr Opin Allergy Clin Immunol, 2006; 6(6):482-8.

The IDO enzyme is well characterized (see, for example, Taylor et al., FASEB Journal 1991; 5:2516-2522; Lee et al., Laboratory Investigation, 2003; 83:1457-1466; and Grohmann et al., Trends in Immunology 2003; 24:242-248) and compounds that serve as substrates or inhibitors of the IDO enzyme are known. For example, Southan (Southan et al, Med. Chem Res., 1996; 343-352) utilized an in vitro assay system to identify tryptophan analogues that serve as either substrates or inhibitors of human IDO. Methods for detecting the expression of IDO in cells are well known and include, but are not limited to, any of those described herein and those described, for example in U.S. Pat. Nos. 6,395,876, 6,451,840, and 6,482,416, US. Patent Application Nos. 20030194803, 20040234623, 20050186289, and 20060292618, and PCT application “The Induction of Indoleamine 2,3-dioxygenase in Dendritic Cells by TLR Ligands and Uses Thereof,” filed Oct. 21, 2006.

IDO degrades the essential amino acid tryptophan (for reviews see Taylor et al., FASEB Journal 1991; 5:2516-2522; Lee et al., Laboratory Investigation, 2003; 83:1457-1466; and Grohmann et al., Trends in Immunology 2003; 24:242-248). IDO is the first and rate-limiting step in the degradation of tryptophan to the downstream metabolite kynurenine (KYN) and subsequent metabolites along the KYN pathway (Mellor and Munn, Nat Rev Immunol 2004; 4:762-774; Grohmann et al., Trends Immunol 2003; 24:242-248). IDO mediates T cell regulatory effects in inflammatory conditions associated with a diverse range of clinical syndromes including cancer, infectious and autoimmune diseases, allergy and tissue transplantation and pregnancy, (Munn et al., Science 1998; 281:1191-1193; Gurtner et al., Gastroenterology 2003; 125:1762-1773; Uyttenhove et al., Nat Med 2003; 9:1269-1274; Muller et al., Nat Med 2005; 11:312-319; Munn et al., J Clin Invest 2004; 114:280-290; Swanson et al., Am. J Respir Cell Mol Biol 2004; 30:311-318; Hayashi et al., J Clin Invest 2004; 114:270-279; Potula et al., Blood 2005; 106:2382-2390).

Expression of IDO by human monocyte-derived macrophages (Munn et al., J. Exp. Med. 1999; 189:1363-1372), human dendritic cells (Munn et al., Science 2002; 297:1867-1870 and Hwu et al., J Immunol. 2000; 164:3596-3599), and mouse dendritic cells (Mellor et al., J. Immunol. 2003; 171:1652-1655) allows these different antigen-presenting cells (APCs) to inhibit T cell proliferation in vitro. In vivo, IDO participates in maintaining maternal tolerance toward the antigenically foreign fetus during pregnancy (Munn et al., Science 1998; 281:1191-1193).

IDO has also been implicated in maintaining tolerance to self antigens (Grohmann et al., J. Exp. Med. 2003; 198:153-160), in suppressing T cell responses to MHC-mismatched organ transplants (Miki et al., Transplantation Proceedings 2001; 33:129-130), and in the tolerance-inducing activity of recombinant CTLA4-Ig (Grohmann et al., Nature Immunology 2002; 3:985-1109). In these three systems, the immunosuppressive effect of IDO can be blocked by the in vivo administration of an IDO inhibitor, such as 1-methyl-tryptophan (also referred to herein as 1-MT or 1MT). In mice, IDO is expressed in certain DC subsets, including PDCs, that have been linked to immunosuppression and tolerance induction (Grohmann et al., 2001 J Immunol 167:708-714; Mellor et al., 2003 J Immunol 171:1652-1655; and Munn et al., 2004 J Clin Invest 114:280-29010-12).

The transfection of IDO into mouse tumor cell lines confers the ability to suppress T cell responses both in vitro and in vivo (Mellor et al., J. Immunol. 2002; 168:3771-3776). In a Lewis Lung carcinoma (LLC) model, administration of 1-MT significantly delayed tumor outgrowth (Friberg et al., International Journal of Cancer 2002; 101:151-155). The mouse mastocytoma tumor cell P815 line forms lethal tumors in naive hosts, but is normally rejected by pre-immunized hosts. However, transfection of P815 with IDO prevents its rejection by pre-immunized hosts (Uyttenhove et al., Nature Medicine 2003; 9:1269-1274). This effect was entirely dependent on the presence of an intact immune system and was substantially reversed, that is, tumor growth inhibited, by the concomitant administration of 1-MT.

The present invention includes methods of suppressing the generation of Tregs, reducing the immune suppression mediated by Tregs, reducing the induction of antigen-specific Tregs, and/or enhancing an immune response to an antigen by administering an inhibitor of IDO.

IDO inhibitors include, but are not limited to, 1-methyl-tryptophan, β-(3 benzofuranyl)-alanine, β-[3-benzo(b)thienyl]-alanine, 6-nitro-tryptophan, and derivatives thereof. An inhibitor of IDO may be an L isomer, a D isomer, or a racemic mixture of IDO. In some embodiments, a preferred IDO inhibitor is 1-methyl-tryptophan, also referred to as 1MT or 1-MT. In some embodiments, an IDO inhibitor is a D isomer of 1MT, an L isomer of 1MT, or a racemic mixture of 1MT. See, for example, published U.S. Patent Application Nos. 2004/0234623 and 2005/0186289. Additional examples of compounds that inhibit IDO activity are brassinin derivatives described by Gaspari et al., J Medicinal Chem 2006; 49(2):684-92), a series of indole derivatives described in patent application PCT/US04/05154, and a series of compounds derived from naphtoquinones described in WO/2006/005185. Inhibitors of the IDO enzyme are readily commercially available, for example, from Sigma-Aldrich Chemicals, St. Louis, Mo.

Additional examples of compounds that inhibit IDO activity include, for example, any of the compounds with IDO inhibitory activity described in Prendergast et al., “Novel Indoleamine-2,3-dioxygenase inhibitors,” (PCT/US2004/005154); Peterson et al., “Evaluation of substituted beta-carbolines as noncompetitive indoleamine-2,3-dioxygenase inhibitors,” (Med Chem Res 1993; 3:473-482); Gaspari et al., “Structure-activity study of brassinin derivatives as indoleamine-2,3-dioxygenase inhibitors,” (J. Med. Chem 2006; 49:684-92); Vottero et al., “Inhibitors of human indoleamine 2,3 dioxygenase identified with a target-based screen in yeast,” (Biotechnol J. 2006; 1:282-288); Sono et al., “Heme containing oxygenases,” Chem Rev 1996; 96:2841); Muller et al., “Inhibition of indoleamine 2,3-dioxygenase an immunoregulatory target of the cancer suppression gene Bin1 potentiates cancer immunotherapy,” Nat. Med 2005; 11:312-319); Peterson et al., “Evaluation of substituted beta-carbolines as noncompetitive indoleamine-2,3-dioxygenase inhibitors,” Med Chem Res 1993; 4:473-482); Sono et al., “Enzyme kinetic and spectroscopic studies of inhibitor and effector interactions with indoleamine-2,3-dioxygenase,” (Biochemistry 1989; 28:5392-9); and Andersen et al., “Indoleamine-2,3-dioxygenase inhibitors,” (PCT/CA2005/001087). For example, inhibitors include any of A-YY, shown below, and analogs and derivatives thereof, wherein an analog or derivative thereof inhibits IDO.

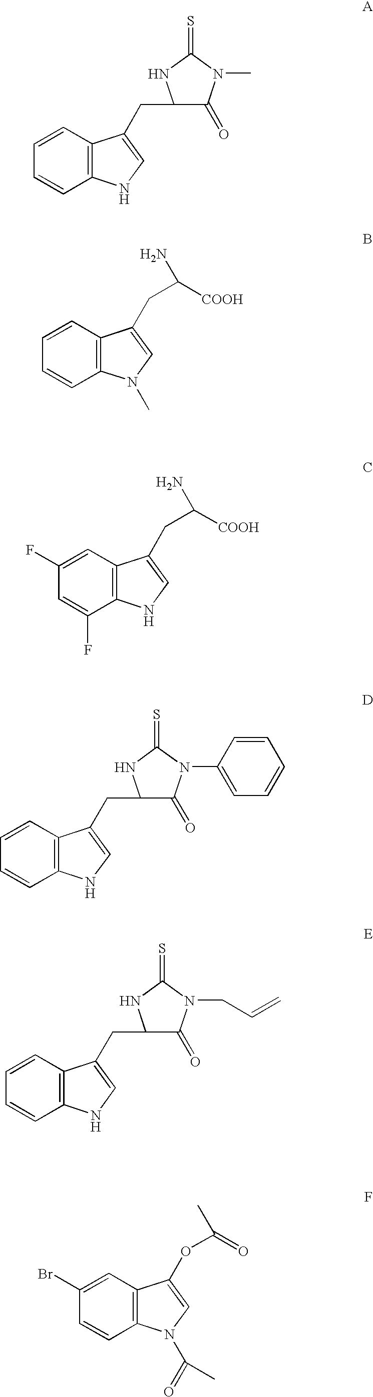

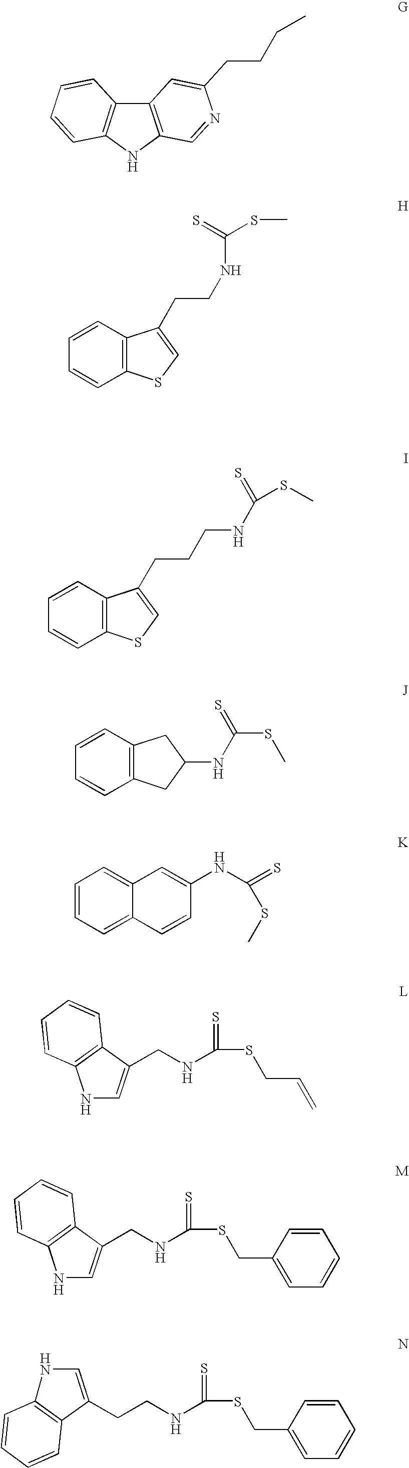

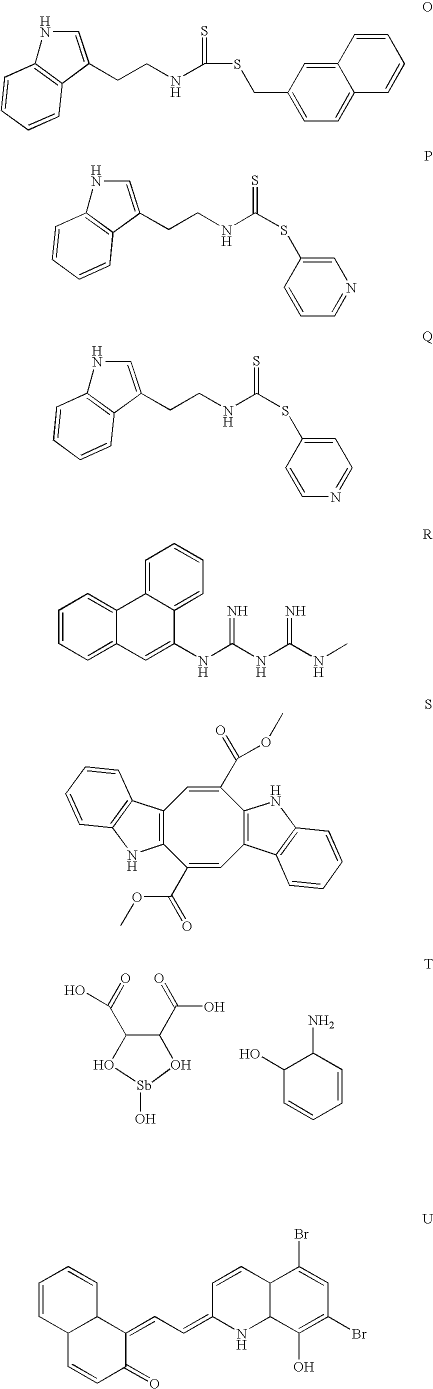

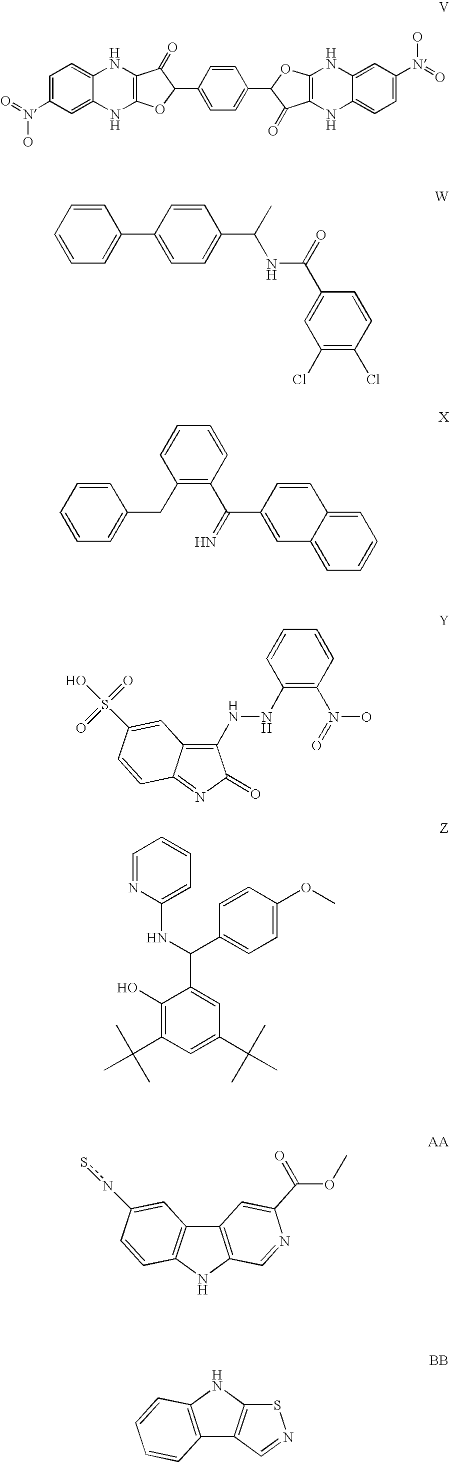

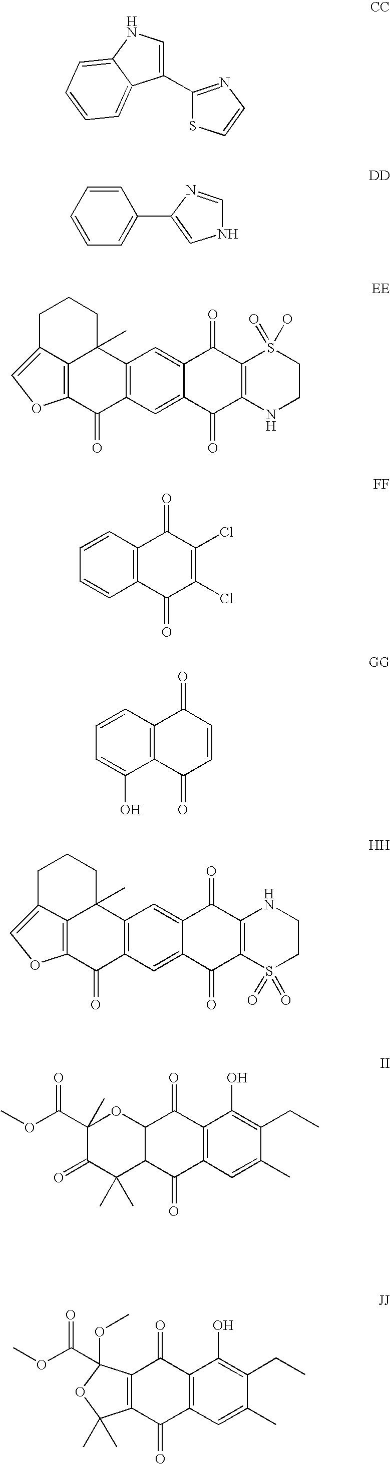

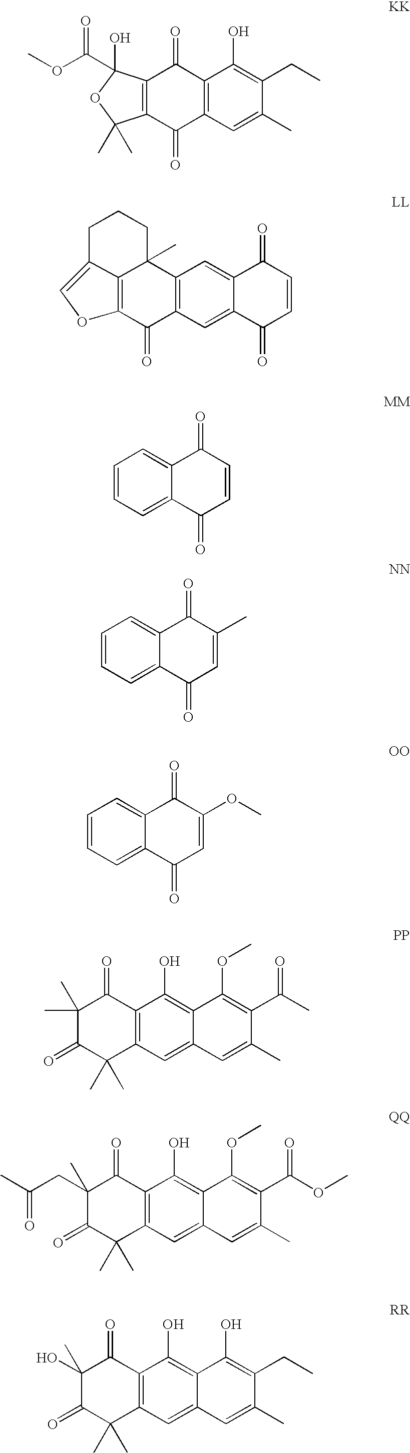

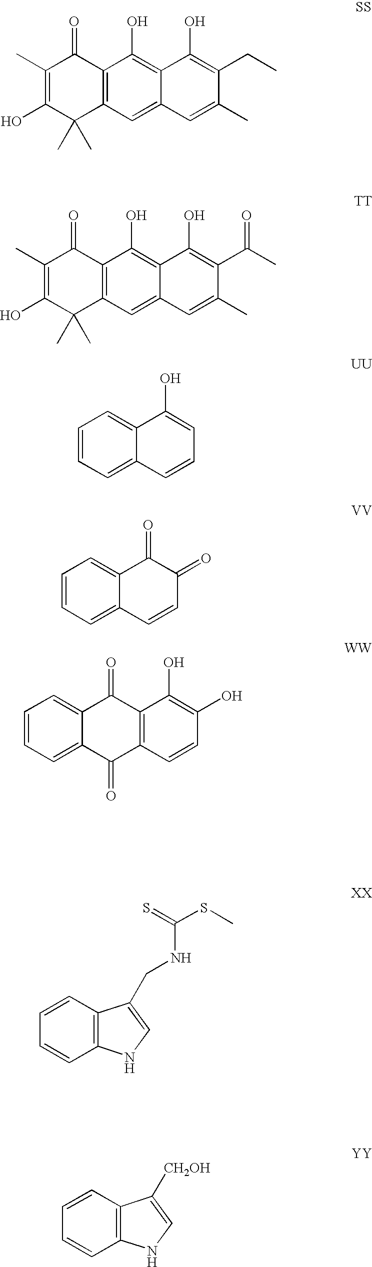

Inhibitor A has an EC50 of approximately 12-20 μM and a Ki of approximately 11 μM (Prendergast et al., PCT/US2004/005154; Muller et al., Nat. Med 2005; 11:312-319). Inhibitor B has an EC50 of approximately 35-50 μM and a Ki of approximately 6-34 μM (Prendergast et al., PCT/US2004/005154). Inhibitor C has a Ki of approximately 24 μM (Sono et al., Chem Rev 1996; 96:2841). Inhibitor D has an EC50 of approximately 100 μM; inhibitor E has an EC50 of approximately 50 μM; and inhibitor F has an EC50 of approximately 200 μM (Prendergast et al., PCT/US2004/005154). Inhibitor G has a Ki of approximately 3 μM (Peterson et al., Med Chem Res 1993; 3:473-482). Inhibitor H has a Ki of approximately 41 μM; inhibitor I has a Ki of approximately 34 μM; inhibitor J has a Ki of approximately 42 μM; inhibitor K has a Ki of approximately 47 μM; inhibitor L has a Ki of approximately 37 μM; inhibitor M has a Ki of approximately 13 μM; inhibitor N has a Ki of approximately 17 μM; inhibitor O has a Ki of approximately 11 μM; inhibitor P has a Ki of approximately 28 μM; and inhibitor Q has a Ki of approximately 20 μM (Gaspari et al., J. Med. Chem 2006; 49:684-92). Inhibitor R has an EC50 of approximately 3 μM and a Ki of approximately 1.5 μM; inhibitor S has an EC50 of approximately 1 μM; inhibitor T has an EC50 of approximately 5 nM; inhibitor U has an EC50 of approximately 1 μM; inhibitor V has an EC50 of approximately 1 μM; inhibitor W has an EC50 of approximately 2 μM; inhibitor X has an EC50 of approximately 5 μM; inhibitor Y has an EC50 of approximately 5 μM; and inhibitor Z has an EC50 of approximately 6 μM (Vottero et al., Biotechnol J. 2006; 1:282-288). Inhibitor AA has a Ki of approximately 8.5 μM; inhibitor BB has a Ki of approximately 5 μM; and inhibitor CC (Peterson et al., Med Chem Res 1993; 4:473-482). Inhibitor DD has a Ki of approximately 4 μM (Sono et al., Biochemistry 1989; 28:5392-9). Inhibitor EE has a Ki of approximately 25 nM; inhibitor FF has a Ki of approximately 45 nM; inhibitor GG has a Ki of approximately 48 nM; inhibitor HH has a Ki of approximately 86 nM; inhibitor II has a Ki of approximately 120 nM; inhibitor JJ has a Ki of approximately 140 nM; inhibitor KK has a Ki of approximately 0.6 μM; inhibitor LL has a Ki of approximately 180 nM; inhibitor MM has a Ki of approximately 0.3 μM; inhibitor NN has a Ki of approximately 0.6 μM; inhibitor OO has a Ki of approximately 0.5 μM; inhibitor PP has a Ki of approximately 1.2 μM; and inhibitor QQ has a Ki of approximately 1.2 μM (Andersen et al., PCT/CA2005/001087). Inhibitor RR has a Ki of approximately 1.4 μM; inhibitor SS has a Ki of approximately 3.1 μM; inhibitor TT has a Ki of approximately 3.2 μM; inhibitor UU has a Ki of approximately 1.8 μM; inhibitor VV has a Ki of approximately 3.4 μM; and inhibitor WW has a Ki of approximately 42 μM. Inhibitor XX has an EC50 of approximately 100 μM and a Ki of approximately 97 μM (Prendergast et al., PCT/US2004/005154; Gaspari et al., J. Med. Chem 2006; 49:684-92). Inhibitor YY has an EC50 of approximately 100 μM (Prendergast et al., PCT/US2004/005154).

The present invention demonstrates that IDO expression is necessary for the generation of CD4+ Tregs and demonstrates that this effect can be pharmacologically reproduced by the addition of a metabolic breakdown product of tryptophan, or an analog of a metabolic breakdown product of tryptophan. Tryptophan is also referred to herein as “Tryp,” “tryp,” “Trp” or “trp.” IDO degrades the essential amino acid tryptophan (Trp) to kynurenin (KYN), which is then metabolized by other enzymes to subsequent metabolites along the KYN pathway (Stone and Darlington, Nat Rev Drug Discov 2002; 1:609-620). The present invention includes the administration of a metabolic breakdown product of tryptophan, or an analog of a metabolic breakdown product of tryptophan, for the generation of Tregs. As used herein, an “analog” refers to a chemical compound or molecule made from a parent compound or molecule by one or more chemical reactions. As such, an analog can be a compound with a structure similar to or based on that of a metabolic breakdown product of tryptophan, but differing from it in respect to certain components or structural makeup, which may have a similar action metabolically. In preferred embodiments, the metabolic breakdown product of tryptophan is L-kynurenine, kynurenic acid, anthranilic acid, 3-hydroxyanthranilic acid, quinolinic acid, or picolinic acid, and an analog of a metabolic breakdown product of tryptophan is an analog of L-kynurenine, kynurenic acid, anthranilic acid, 3-hydroxyanthranilic acid, quinolinic acid, or picolinic acid. A metabolic breakdown product of tryptophan, or an analog of a metabolic breakdown product of tryptophan, may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

With the present invention, an agonist of one or more Toll-like receptors (TLRs) may be administered to a subject to induce the generation of Tregs. The terms “agonist” and “agonistic,” as used herein, refer to or describe an agent that is capable of substantially inducing, promoting or enhancing TLR biological activity or TLR receptor activation or signaling. The terms “antagonist” or “antagonistic,” as used herein, refer to or describe an agent that is capable of substantially counteracting, reducing or inhibiting TLR biological activity or TLR receptor activation or signaling. In some aspects of the present invention, a TLR9 agonist may be administered to induce the expression of IDO. As used herein, a TLR9 agonist refers to an agent that is capable of substantially inducing, promoting or enhancing TLR9 biological activity or TLR9 receptor activation or signaling. TLR9 is activated by unmethylated CpG-containing sequences, including those found in bacterial DNA or synthetic oligonucleotides (ODNs). A TLR9 agonist may be a preparation of microbial DNA, including, but not limited to, E. coli DNA, endotoxin free E. coli DNA, or endotoxin-free bacterial DNA from E. coli K12. A TLR9 agonist may be isolated from a bacterium, for example, separated from a bacterial source; synthetic, for example, produced by standard methods for chemical synthesis of polynucleotides; produced by standard recombinant methods, then isolated from a bacterial source; or a combination of the foregoing.

In preferred embodiments, a TLR9 agonist is a synthetic oligonucleotide containing unmethylated CpG motifs, also referred to herein as “a CpG-oligodeoxynucleotide,” “CpGODNs,” or “ODN” (see, for example, Hemmi et al., Nature 2000; 408:740-745). A CpG-oligodeoxynucleotide TLR9 agonist includes a CpG motif. A CpG motif includes two bases to the 5′ and two bases to the 3′ side of the CpG dinucleotide. CpG-oligodeoxynucleotides may be produced by standard methods for chemical synthesis of polynucleotides. CpG-oligodeoxynucleotides may be purchased commercially, for example, from Coley Pharmaceuticals (Wellesley, Mass.), Axxora, LLC (San Diego, Calif.), or InVivogen, (San Diego, Calif.). A CpG-oligodeoxynucleotide TLR9 agonist may includes a wide range of DNA backbones, modifications and substitutions. In some aspects of the invention, a TLR9 agonist is a nucleic acid that includes the nucleotide sequence 5′ CG 3′. In some aspects of the invention, a TLR9 agonist is a nucleic acid that includes the nucleotide sequence 5′-purine-purine-cytosine-guanine-pyrimidine-pyrimidine-3′. In other aspects of the invention, a TLR9 agonist is a nucleic acid that includes the nucleotide sequence 5′-purine-TCG-pyrimidine-pyrimidine-3′. In some aspects of the invention, a TLR9 agonist is a nucleic acid that includes the nucleotide sequence 5′-(TGC)ₙ-3′, where n≧1. In other aspects of the invention, a TLR9 agonist is a nucleic acid that includes the sequence 5′-TCGNN-3′, where N is any nucleotide.

With the methods of the present invention, a TLR agonist may be administered at a low dosage. In human subjects, a low dosage of a CpG agonist is about 30 mg or less. A low dosage of a CpG agonist may be about 25 mg or less. A low dosage of a CpG agonist may be about 20 mg or less. A low dosage of a CpG agonist may be about 15 mg or less. A low dosage of a CpG agonist may be about 10 mg or less. A low dosage of a CpG agonist may be about 5 mg or less. A low dosage of a CpG agonist may be about 1 mg or less. A low dosage of a CpG agonist may be about 0.5 mg or less. A low dosage of a CpG agonist may be a range of any of these dosages. For example, a low dosage of a CpG agonist may be from about 0.5 mg to about 30 mg. Such a low dosage may be administered, for example, when a TLR agonist is administered as a vaccine adjuvant. Such a low dosage may, for example, be administered subcutaneously, intradermal, or intratumoral.

With the methods of the present invention, a TLR agonist may be administered at a high dosage. In human subjects a high dosage is greater than 30 mg. A high dosage may, for example, be greater than about 30 mg, greater than about 50 mg, greater than about 75 mg, greater than about 100 mg, greater than about 125 mg, greater than about 150 mg, or more. A high dosage may be up to about 125 mg, up to about 250 mg, up to about 500 mg, or more. Such a high dosage maybe administered, for example, to induce an immunosuppressive effect. Such a low dosage may be administered systemically, including, for example, intravenously.

A TLR agonist may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

The methods of the present invention may also be administered to a patient receiving a vaccine. Such a vaccine may be an anti-viral vaccine, such as, for example, a vaccine against HIV, or a vaccine against tuberculosis or malaria. The vaccine may be a tumor vaccine, including, for example, a melanoma, prostate cancer, colorectal carcinoma, or multiple myeloma vaccine. Dendritic cells (DC) have the ability to stimulate primary T cell antitumor immune responses. Thus, a tumor vaccine may include dendritic cells. Dendritic cell vaccines may be prepared, for example, by pulsing autologous DCs derived from the subject with synthetic antigens, tumor lysates, tumor RNA, or idiotype antibodies, by transfection of DCs with tumor DNA, or by creating tumor cell/DC fusions (Ridgway, Cancer Invest. 2003; 21:873-86). The vaccine may include one or more immunogenic peptides, for example, immunogenic HIV peptides, immunogenic tumor peptides, or immunogenic human cytomegalovirus peptides (such as those described in U.S. Pat. No. 6,251,399). The vaccine may include genetically modified cells, including genetically modified tumor cells or cell lines genetically modified to express granulocyte-macrophage stimulating factor (GM-CSF) (Dranoff, Immunol Rev. 2002; 188:147-54). In some aspects of the invention, a vaccine may include an antigen that is the target of an autoimmune response.

The methods of the present invention may be used in the treatment of an autoimmune disease. Autoimmune diseases that may be treated by the methods of the present invention include, but are not limited to, acute disseminated encephalomyelitis (ADEM), Addison's disease, ankylosing spondylitisis, antiphospholipid antibody syndrome (APS), aplastic anemia, autoimmune hepatitis, autoimmune uveitis celiac disease, Crohn's disease, Goodpasture's syndrome, Graves' disease, Guillain-Barré syndrome (GBS), Hashimoto's disease, idiopathic thrombocytopenic purpura, insulin dependent diabetes mellitus (IDDM) lupus erythematosus, multiple sclerosis, myasthenia gravis, opsoclonus myoclonus syndrome (OMS), Ord's thyroiditis, pemphigus, pernicious Anaemia, polyarthritis, primary biliary cirrhosis, rheumatoid arthritis, Reiter's syndrome, Sjögren's syndrome, Takayasu's arteritis, temporal arteritis (also known as giant cell arteritis), warm autoimmune hemolytic anemia, and Wegener's granulomatosi.

Certain pathological conditions, such as parasitic infections, AIDS (caused by the human immunodeficiency virus (HIV)) and latent cytomegaloviral (CMV) infections, are extremely difficult to treat since the macrophages act as reservoirs for the infectious agent. Even though the cells are infected with by a foreign pathogen, they are not recognized as foreign. The methods of the present invention may be used to treat such pathological conditions including, but not limited to, viral infections, infection with an intracellular parasite, and infection with an intracellular bacteria. Viral infections treated include, but are not limited to, infections with the human immunodeficiency virus (HIV) or cytomegalovirus (CMV). Intracellular bacterial infections treated include, but are not limited to infections with Mycobacterium leprae, Mycobacterium tuberculosis, Listeria monocytogenes, and Toxplasma gondii. Intracellular parasitic infections treated include, but are not limited to, Leishmania donovani, Leishmania tropica, Leishmania major, Leishmania aethiopica, Leishmania mexicana, Plasmodium falciparum, Plasmodium vivax, Plasmodium ovale, and Plasmodium malariae. The efficacy of treatment of an infection may be assessed by any of various parameters well known in the art. This includes, but is not limited to, a decrease in viral load, an increase in CD4⁺ T cell count, a decrease in opportunistic infections, eradication of chronic infection, and/or increased survival time.

Current experimental methods of cancer treatment include tumor vaccination protocols including the administration of tumor peptides or whole cell tumor vaccines with CpG ODNs as immunostimulatory adjuvants. Currently CpG ODNs have been utilized as an adjuvant along with a tumor vaccine. However, as shown by the present invention, the administration of a CpG ODN adjuvant can induce the expression of IDO in a subpopulation of DCs that may lead to partial or full immunosuppression, precluding the full immunostimulatory capacity of DCs and therefore potentially dampening the immune response to tumor specific antigens. The present invention provides methods to enhance the immunostimulatory capacity of DCs to tumor antigens by co-administration of one or more inhibitors of IDO along with the administration of a TLR agonist, in an amount effective to suppress the induction of Tregs. The present invention includes methods of treating cancer in a subject by administering to the subject an inhibitor of IDO in an amount effective to suppress the induction or Tregs. The present invention also includes methods of treating cancer in a subject by administering an inhibitor of IDO along with a TLR agonist, such as, for example, a CpG oligonucleotide and/or an inhibitor of GCN2 and/or additional therapeutic treatments in an amount effective to suppress the induction or Tregs. Additional therapeutic treatments include, but are not limited to, surgical resection, radiation therapy, chemotherapy, hormone therapy, anti-tumor vaccines, antibody based therapies, whole body irradiation, bone marrow transplantation, peripheral blood stem cell transplantation, and the administration of chemotherapeutic agents (also referred to herein as “antineoplastic chemotherapy agent”). Antineoplastic chemotherapy agents include, but are not limited to, cyclophosphamide, methotrexate, 5-fluorouracil, doxorubicin, vincristine, ifosfamide, cisplatin, gemcitabine, busulfan (also known as 1,4-butanediol dimethanesulfonate or BU), ara-C (also known as 1-beta-D-arabinofuranosylcytosine or cytarabine), adriamycin, mitomycin, cytoxan, methotrexate, and combinations thereof. Additional therapeutic agents include, for example, one or more cytokines, an antibiotic, antimicrobial agents, antiviral agents, such as AZT, ddI or ddC, and combinations thereof. The cytokines used include, but are not limited to, IL-1α, IL-1β, IL-2, IL-3, IL-4, IL-6, IL-8, IL-9, IL-10, IL-12, IL-18, IL-19, IL-20, IFN-α, IFN-β, IFN-γ, tumor necrosis factor (TNF), transforming growth factor-β (TGF-β), granulocyte colony stimulating factor (G-CSF), macrophage colony stimulating factor (M-CSF), granulocyte-macrophage colony stimulating factor (GM-CSF)) (U.S. Pat. Nos. 5,478,556, 5,837,231, and 5,861,159), or Flt-3 ligand (Shurin et al., Cell Immunol. 1997; 179:174-184). Antitumor vaccines include, but are not limited to, peptide vaccines, whole cell vaccines, genetically modified whole cell vaccines, recombinant protein vaccines or vaccines based on expression of tumor associated antigens by recombinant viral vectors.

The tumors to be treated by the present invention include, but are not limited to, melanoma, colon cancer, pancreatic cancer, breast cancer, prostate cancer, lung cancer, leukemia, lymphoma, sarcoma, ovarian cancer, Kaposi's sarcoma, Hodgkin's Disease, Non-Hodgkin's Lymphoma, multiple myeloma, neuroblastoma, rhabdomyosarcoma, primary thrombocytosis, primary macroglobulinemia, small-cell lung tumors, primary brain tumors, stomach cancer, malignant pancreatic insulinoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

The efficacy of treatment of a tumor may be assessed by any of various parameters well known in the art. This includes, but is not limited to, determinations of a reduction in tumor size, determinations of the inhibition of the growth, spread, invasiveness, vascularization, angiogenesis, and/or metastasis of a tumor, determinations of the inhibition of the growth, spread, invasiveness and/or vascularization of any metastatic lesions, and/or determinations of an increased delayed type hypersensitivity reaction to tumor antigen. The efficacy of treatment may also be assessed by the determination of a delay in relapse or a delay in tumor progression in the subject or by a determination of survival rate of the subject, for example, an increased survival rate at one or five years post treatment. As used herein, a relapse is the return of a tumor or neoplasm after its apparent cessation, for example, such as the return of leukemia.

The present invention also includes methods of preventing graft versus host disease (GVHD) in a recipient, the method including administering to a the donor a metabolic breakdown product of tryptophan, or an analog of a metabolic breakdown product of tryptophan, and one or more alloantigens present in the recipient, wherein the a metabolic breakdown product of tryptophan, or an analog of a metabolic breakdown product of tryptophan, and the one or more alloantigens present in the recipient are administered to the donor prior to obtaining donor cells from the donor; obtaining donor cells from the donor; and administering the donor cells to the recipient. GVHD is a complication of an allogeneic bone marrow or cord blood transplant (BMT) in which functional immune cells in the transplanted marrow recognize the recipient as “foreign” and mount an immunologic attack. Thus, GVHD is a pathological condition in which cells from the transplanted tissue of a donor initiate an immunologic attack on the cells and tissue of the recipient. After bone marrow transplantation, T cells present in the graft, either as contaminants or intentionally introduced into the host, attack the tissues of the transplant recipient after perceiving host tissues as antigenically foreign. A wide range of host antigens, also referred to herein as “alloantigens” can initiate GVHD, among them the HLAs. However, graft-versus-host disease can occur even when HLA-identical siblings are the donors. HLA-identical siblings or HLA-identical unrelated donors (called a minor mismatch as opposed to differences in the HLA antigens, which constitute a major mismatch) often still have genetically different proteins that can be presented on the MHC.

The present invention includes methods of preconditioning a recipient of an allograft to suppress allograft rejection in the recipient, the method including administering to the recipient a metabolic breakdown product of tryptophan, or an analog of a metabolic breakdown product of tryptophan, and one or more alloantigens present in the allograft, wherein the metabolic breakdown product of tryptophan, or an analog of a metabolic breakdown product of tryptophan, and the one or more alloantigens present in the allograft are administered to the recipient prior to allografting; and transplanting the allograft into the recipient.

The present invention includes isolated cell populations preconditioned to minimize graft versus host disease when transplanted into a recipient. The cell populations may be obtained by administering to the donor a metabolic breakdown product of tryptophan, or an analog of a metabolic breakdown product of tryptophan, and one or more alloantigens present in the recipient, wherein the metabolic breakdown product of tryptophan, or an analog of a metabolic breakdown product of tryptophan, and the one or more alloantigens present in the recipient are administered to the donor prior to obtaining donor cells from the donor; and obtaining donor cells from the donor. The metabolic breakdown product of tryptophan, or an analog of a metabolic breakdown product of tryptophan, may be administered in an amount effective to induce IDO expression in an IDO-competent subset of DCs. The metabolic breakdown product of tryptophan, or an analog of a metabolic breakdown product of tryptophan, may be administered in an amount effective to induce IDO expression in subpopulation of splenic DCs. Such preconditioned cell populations can be used in a number of immunotherapies, including, for example, for the prevention of GVHD, to decrease the likelihood of rejection of an allograft or xenotransplanted tissue or organ, or the treatment of autoimmune diseases.

The present invention includes the use of inhibitors of GCN2 to prevent the development or reactivation of Tregs by IDO. The protein kinase GCN2 (also referred to as “General Control Nonderepressible 2,” “eIF2AK4,” and “eukaryotic translation initiation factor 2 alpha kinase 4”) has been shown to play a role in the induction of proliferative arrest and anergy of CD8+ T cells in the presence of IDO+ DCs (see Munn et al., Immunity 2005; 22:1-10). Specifically, Munn et al. demonstrated that in order for IDO to mediate the proliferative arrest and anergy of effector T cells, the cells need GCN2. Thus, GCN2 is downstream in the pathway of IDO effects and inhibiting the function of GCN2 with an inhibitory agent should result in blockade of the inhibitory effect of IDO on the effector T cells. Example 1 describes that the expression of IDO by human DCs induces the differentiation of naïve CD4+ T cells into Tregs, and that this is mediated by Trp metabolites such as Kynurenine. It has also been shown that the combined effects of Trp depletion and Trp catabolites induces naïve T cells to acquire a regulatory phenotype, and that this mechanism was mediated by GCN2, since T cells from GCN2 knockout animals did not develop the regulatory phenotype (Fallarino et al., J Immunol 2006; 176:6752-6761). Examples 2 and 3 provide evidence showing that reactivation of pre-existing Tregs by IDO expressed in DCs requires GCN2. Thus, targeting GCN2 kinase with inhibitory agents can serve as an alternative to direct IDO inhibition (see, also, Muller and Scherle, Nature Reviews Cancer 2006; 6:613). Thus, GCN2 has been implicated in mediating the effects of IDO in various cell types, including, but not limited to, effector CD8+ T cells and naïve CD4+ T cells. Inhibitors of GCN2 may be used to bypass or replace the need for IDO inhibitors. The present invention includes any of the various methods described herein, in which an IDO inhibitor is replaced by or supplemented with a GCN2 inhibitor. Candidate GCN2 inhibitors, include, for example, a GCN2 blocking peptide, an antibody to GCN2 (both commercially available, for example, from Bethyl, Inc., Montgomery, Tex.) and small molecule inhibitors (including, for example, those discussed by Muller and Scherle, Nature Reviews Cancer 2006; 6:613).

The present invention includes methods to enhance an immune response in a subject by administering an effective amount of an inhibitor of a GCN2 kinase. With such a method a vaccine may also be administered, either simultaneously or shortly before or after the administration of an inhibitor of GCN2. The present invention includes methods to enhance the immune response in a subject to a vaccine antigen by administering to the subject the vaccine antigen, a CpG oligonucleotide (ODN), and an inhibitor of GCN2. The present invention also includes methods to enhance the immune response in a subject to a vaccine antigen by administering to the subject the vaccine antigen and an inhibitor of GCN2.

The present invention includes methods to prevent immune suppression mediated by Tregs with the administration of an effective amount of an inhibitor of a GCN2 kinase. The present invention also includes methods to enhance an immune response in a subject by administering two or more agents selected from an inhibitor of indoleamine-2,3-dioxygenase (IDO), a CpG oligonucleotide (ODN), an inhibitor of a GCN2 kinase, a vaccine, and/or a chemotherapeutic agent.

The present invention also includes methods to prevent immune suppression mediated by Tregs with the administration of two or more agents selected from an inhibitor of indoleamine-2,3-dioxygenase (IDO), a CpG oligonucleotide (ODN), an inhibitor of a GCN2 kinase, a vaccine, and/or a chemotherapeutic agent.

The present invention includes compositions including one or more inhibitors of GCN2. In some embodiments, such a composition may also include one or more additional active agents, including, for example, one or more IDO inhibitors, one of more TLR agonists, such as for example, one or more CpG oligonucleotides (ODN), one or more antigens, one or more metabolic breakdown products of tryptophan, one or more analogs of a metabolic breakdown product of tryptophan, or one or more chemotherapeutic agents. Chemotherapeutic agents include, for example, an antineoplastic chemotherapy agent, including, but not limited to, cyclophosphamide, methotrexate, fluorouracil, doxorubicin, vincristine, ifosfamide, cisplatin, gemeytabine, busulfan (also known as 1,4-butanediol dimethanesulfonate or BU), ara-C (also known as 1-beta-D-arabinofuranosylcytosine or cytarabine), adriamycin, mitomycin, cytoxan, methotrexate, or a combination thereof. Additional therapeutic agents also include cytokines, including, but not limited to, macrophage colony stimulating factor, interferon gamma, granulocyte-macrophage stimulating factor (GM-CSF), flt-3, an antibiotic, antimicrobial agents, antiviral agents, such as AZT, ddI or ddC, and combinations thereof.

As used herein “treating” or “treatment” includes both therapeutic and prophylactic treatments. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis.

The agents of the present invention can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical, or injection into or around the tumor.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intraperitoneal, and intratumoral administration. In this connection, sterile aqueous media that can be employed will be known to those of skill in the art in light of the present disclosure (see for example, “Remington's Pharmaceutical Sciences” 15th Edition). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA.

For enteral administration, the inhibitor may be administered in a tablet or capsule, which may be enteric coated, or in a formulation for controlled or sustained release. Many suitable formulations are known, including polymeric or protein microparticles encapsulating drug to be released, ointments, gels, or solutions which can be used topically or locally to administer drug, and even patches, which provide controlled release over a prolonged period of time. These can also take the form of implants. Such an implant may be implanted within the tumor.

Therapeutically effective concentrations and amounts may be determined for each application herein empirically by testing the compounds in known in vitro and in vivo systems, such as those described herein, dosages for humans or other animals may then be extrapolated therefrom.

An agent of the present invention may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

With the present invention, the stimulation or inhibition of an immune response may be measured by any of many standard methods well known in the immunological arts. As used herein, a mixed leukocyte response (MLR) is a well-known immunological procedure, for example, as described in the examples herein. As used herein, T cell activation by an antigen-presenting cell is measured by standard methods well known in the immunological arts. As used herein, a reversal or decrease in the immunosuppressed state in a subject is as determined by established clinical standards. As used herein, the improved treatment of an infection is as determined by established clinical standards. The determination of immunosuppression mediated by an antigen presenting cell expressing indoleamine-2,3-dioxygenase (IDO) includes the various methods as described in the examples herein.

With the methods of the present invention, the efficacy of the administration of one or more agents may be assessed by any of a variety of parameters well known in the art. This includes, for example, determinations of an increase in the delayed type hypersensitivity reaction to tumor antigen, determinations of a delay in the time to relapse of the post-treatment malignancy, determinations of an increase in relapse-free survival time, determinations of an increase in post-treatment survival, determination of tumor size, determination of the number of reactive T cells that are activated upon exposure to the vaccinating antigens by a number of methods including ELISPOT, FACS analysis, cytokine release, or T cell proliferation assays.

As used herein, the term “subject” includes, but is not limited to, humans and non-human vertebrates. Non-human vertebrates include livestock animals, companion animals, and laboratory animals. Non-human subjects also include non-human primates as well as rodents, such as, but not limited to, a rat or a mouse. Non-human subjects also include, without limitation, chickens, horses, cows, pigs, goats, dogs, cats, guinea pigs, hamsters, mink, and rabbits. As used herein, the terms “subject,” “individual,” “patient,” and “host” are used interchangeably. In preferred embodiments, a subject is a mammal, particularly a human.

As used herein “in vitro” is in cell culture and “in vivo” is within the body of a subject.

As used herein, the term “pharmaceutically acceptable carrier” refers to one or more compatible solid or liquid filler, diluents or encapsulating substances which are suitable for administration to a human or other vertebrate animal.

As used herein, the term “isolated” as used to describe a compound shall mean removed from the natural environment in which the compound occurs in nature. In one embodiment isolated means removed from non-nucleic acid molecules of a cell.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

In some embodiments, an “effective amount” of an agent is an amount that results in a reduction of at least one pathological parameter. Thus, for example, in some aspects of the present invention, an effective amount is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual not treated with the agent.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### The Indoleamine 2,3-dioxygenase Pathway is Essential for Plasmacytoid Dendritic Cell-Induced CD4+ Regulatory T Cell Generation

Human plasmacytoid dendritic cells (PDCs) can prime allogeneic naïve CD4+ T cells to differentiate into CD4+CD25+Foxp3+ regulatory T cells (Tregs). However, the molecular mechanism(s) underlying PDC-induced CD4+ Treg generation is unknown. This example shows that human PDCs express high levels of indoleamine 2,3-dioxygenase (IDO) protein. Triggering Toll-like receptor 9 with CpG oligodeoxynucleotides activates PDCs to sustain IDO expression and upregulate T-cell costimulatory molecules. Blocking IDO activity with its pharmacologic inhibitor 1-methyl-D-tryptophan (1MT) significantly abrogates PDC-induced CD4+ Treg generation and converts to the generation of alloreactive T cells. Adding kynurenine (KYN), an immediate downstream metabolite of tryptophan that is generated by IDO, bypasses the 1MT effect, and restores PDC-induced CD4+ Treg generation. This example demonstrates that the IDO pathway is essential for PDC-induced CD4+ Treg generation, and implicates generation of KYN pathway metabolites as the critical molecular mediator of this process.

### Materials and Methods

PDC, B, and T cell isolation. Human PBMC were isolated under IRB-approved protocols from apheresis products of healthy blood donors (Memorial Blood Centers of Minnesota, Minneapolis, Minn.) by Ficoll-Paque density gradient centrifugation. Plasmascytoid dendritic cells (PDCs) were isolated from PBMC using BDCA-4 cell isolation kits and the MACS system, followed by staining and sorting to collect purified Lin-CD11c-CD123+ PDCs, as reported previously (Moseman et al., J Immunol 2004; 173:4433-4442). CD4+CD45RA+ naïve T cells were isolated from PBMC using CD4 T cell isolation kits followed by positive selection with CD45RA microbeads. The purity of naïve CD4+ T cells was greater than 95% for CD4+CD45RA+ expression and less than 0.5% for CD25+ expression. B cells were isolated from PBMC with CD19 microbeads and the MACS system to greater than 98% purity of CD19+ B cells. All cell isolations kits and microbeads were from Miltenyi Biotec (Bergisch Gladbach, Germany).

Reagents. Phosphorothioate-modified type-A CpG ODN 2216: gGGGACGATCGTCgggggG (SEQ ID NO:2), type-B CpG ODN 2006: tcgtcgttttgtcgttttgtcgtT (SEQ ID NO:3) (sequences are shown 5′-3′; small letters represent phosphorothioate linkage; capital letters represent phosphodiester linkage 3′ of the base; bold represents CpGdinucleotides) were from Integrated DNA Technologies (Coralville, Iowa), diluted in PBS, and used at a final concentration of 1 microgram per milliliter (μg/ml). 1-methyl-D-tryptophan (1MT, Sigma-Aldrich) was used at a final concentration of 250 micromolar (μM). Kynurenine (L-KYN, Sigma-Aldrich) was used at a final concentration of 50 μM.

In vitro priming of naive CD4+ T cells. CD4+CD45RA+ naïve T cells were primed with allogeneic PDCs or irradiated B cells (30 Gy) at a 10:1 ratio (e.g., 2×10⁶ naive CD4+ T cells plus 2×105 PDCs per well in 24-well plates) with ODN 2216 or ODN 2006 present in RPMI 1640 medium supplemented with 10% human AB serum. 1MT and/or KYN were added into CpG ODN-PDC or CpG ODN-B cell mediated naive CD4+ T cell priming cultures as indicated. In some experiments, blocking Abs against CD80, CD86, HLA-DR or the control IgG Ab (R&D Systems, Minneapolis, Minn.) were added to CpG-PDC-mediated naive CD4+ T cell priming cultures at a final Ab concentration ranging from 0.1 to 10 μg/ml. After 7 days, primed T cells in cultures were harvested, assessed for their surface phenotype, intracellular Foxp3 expression, and their function in MLR assays.

Flow Cytometry. Fluorescent antibodies (Abs) against human CD3, CD4, CD11c, CD19, CD25, CD40, CD45, CD45RA, CD45RO, CD80, CD86, CD123, HLA-DR, lineage (Lin) markers, and isotype control Abs were from BD Biosciences (San Diego, Calif.). PE-conjugated anti-human Foxp3 staining set (PCH101) was from eBiosciences (San Diego, Calif.) and used per manufacture's instruction. Mean fluorescence intensity (MFI) and positive cell percentages of stained cells were determined by flow cytometry.

Western blots. Protein lysates were prepared from 2×10⁵ fresh or cultured PDCs or B cells. Western blot was performed with antibody specific for IDO protein

MLR assays. The function of CpG-PDC or CpG-B cell primed CD4+ T cells with or without 1MT and/or KYN were determined by plating the primed T cells at graded doses as responders to irradiated allogeneic PBMC in MLR cultures or as third-party T cells into MLR cultures where freshly purified autologous or allogeneic naive CD4+ T cells were stimulated with irradiated allogeneic PBMC. In all T cell proliferation assays, plates were incubated at 37° C. for 5 days and pulsed with 1 μCi of 3H-thymidine per well for the last 18 hours before harvesting. All determinations were carried out in triplicate and 3H-thymidine incorporation was determined.

Data analysis. Data from experiments are expressed as the mean±SD. Statistical analysis of the results between groups was performed by student's t test. Values of p<0.05 were considered significant.

### Results and Discussion

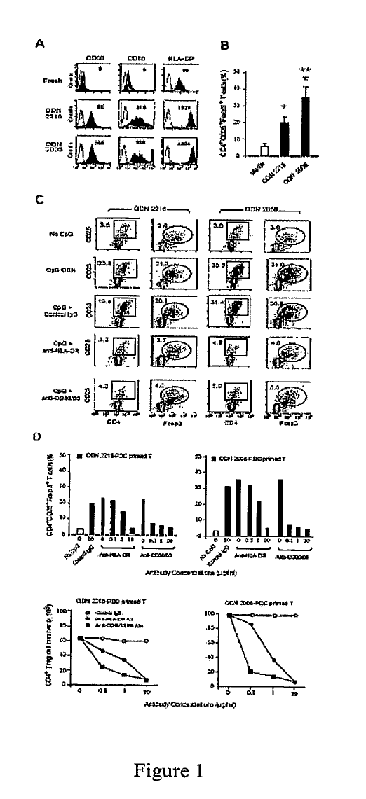

CD4+ Treg generation requires HLA-DR and CD80/86 expression on PDCs. It has been previously shown that CpG ODN promotes PDC-mediated priming of allogeneic naïve CD4+ T cells to differentiate into CD4+CD25+Foxp3+ Tregs (Moseman et al., 2004 J Immunol 173:4433-4442). Freshly isolated human PDCs from peripheral blood express very low levels of T-cell costimulatory molecules such as CD80 and CD86. Triggering TLR9 by type-A (2216) or type-B (2006) CpG ODN rapidly activates PDCs to upregulate cell surface expression of CD80, CD86 molecules and HLA-DR antigens (FIG. 1A). The addition of ODN 2216 or ODN 2006 significantly increases the frequency of PDC-induced CD4+CD25+Foxp3+ Tregs from 5.7±3.1% to 21.6±5.2% or 34.2±7.8%, respectively, at day 7 of cultures (FIG. 1B). Direct cell contact between PDCs and naïve CD4+ T cells is required for the induction of CD4+ Tregs (Moseman et al., 2004 J Immunol 173:4433-4442). PDCs are known to express HLA-DR molecules, which provide a TCR signal to the allogeneic CD4+ T cells. The upregulated expression of B7 ligands (CD80, CD86) on PDCs following CpG ODN stimulation may serve as critical second signal to promote PDC-induced CD4+ Treg generation. To test this hypothesis, experiments were performed by adding graded concentrations of antibodies (Abs) against CD80/CD86 or HLA-DR into the priming cultures of PDCs and allogeneic naïve CD4+ T cells.

Both Abs against CD80/CD86 or HLA-DR antigens effectively abrogated the capability of CpG ODN-activated PDCs (CpG-PDCs) to induce CD4+CD25+Foxp3+ Tregs, whereas control IgG Ab had no significant effect on CpG-PDC-induced CD4+ Tregs (FIG. 1C). The blocking effects of anti-CD80/CD86 Abs or anti-HLA-DR Ab on the frequency and number of CpG-PDC-induced Tregs were Ab dose-dependent (FIG. 1D). These findings demonstrate that PDC-mediated allogeneic CD4+ Treg generation is dependent upon CD4+ T cell signals delivered by MHC class II antigens and costimulation via B7 ligands.

It has been suggested that immature DCs prime T cells to differentiate into suppressor/regulatory T cells, whereas mature DCs prime T cells for an effector-type immune response. However, this example demonstrates that CpG ODN-activated PDCs are phenotypically mature, yet remain tolerogenic and can induce CD4+ Tregs. Therefore, the capacity of PDCs to induce Tregs could not be attributed to their maturation stage, but rather to some intrinsic property of PDCs.

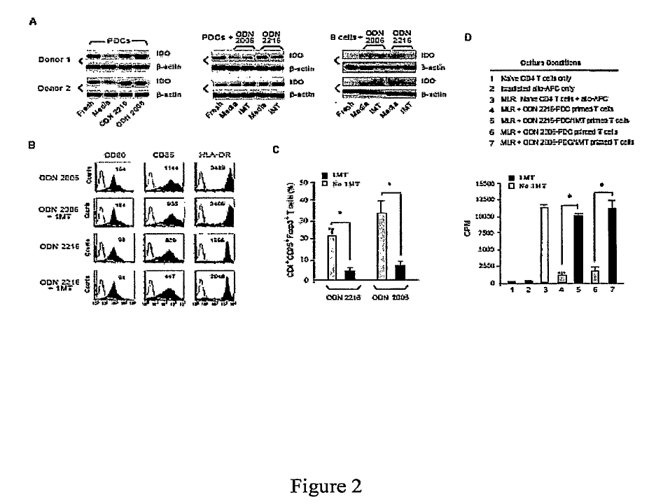

PDCs employ the IDO pathway to induce CD4+ Treg generation. Recent studies have highlighted the role of IDO as a potential mechanism of tolerance and immunosuppression (Mellor and Munn, 2004 Nat Rev Immunol 4:762-774; and Grohmann et al., 2003 Trends Immunol 24:242-248). However, it was not known whether human PDCs expressed IDO, or used the IDO pathway of immunosuppression. Western blots using antibody against an N-terminal peptide of human IDO (Munn et al., Science 2002; 297:1867-1870) demonstrated that freshly isolated human PDCs expressed readily detectable levels of IDO protein (FIG. 2A), at levels much higher than the control cell type (freshly isolated human B cells). TLR9 signaling with either ODN 2216 or ODN 2006 activated PDCs and sustained their IDO expression during culture, whereas PDCs cultured in media alone became apoptotic and dramatically decreased their IDO expression. In contrast, CpG ODN-stimulated human B cells expressed barely detectable levels of IDO (FIG. 2A).

To determine if IDO was mechanistically involved in generation of Tregs by PDCs, 1-methyl-D-tryptophan (1MT), a pharmacologic inhibitor of IDO enzymatic activity, was added to MLRs containing CpG ODN, PDCs plus naive allogeneic CD4+ T cells. It has been previously shown that CD4+ T cells primed in this system acquire characteristics of Tregs, being hyporesponsive to secondary alloantigen stimulation and strongly inhibiting the proliferation of autologous or allogeneic CD4+ T cells in secondary MLR cultures (Moseman et al., 2004 J Immunol 173:4433-4442). The addition of 1MT to priming MLRs had insignificant effects on the expression of cell surface maturation markers by PDCs (CD80, CD86, HLA-DR) (FIG. 2B). However, the addition of 1MT significantly reduced the CD4+CD25+Foxp3+ cells induced by PDCs in the priming MLRs (FIG. 2C). At the functional level, the addition of 1MT blocked the generation of suppressor/regulatory activity of PDC primed T cells to inhibit the proliferation of naïve CD4+ T cells in MLR cultures (FIG. 2D).

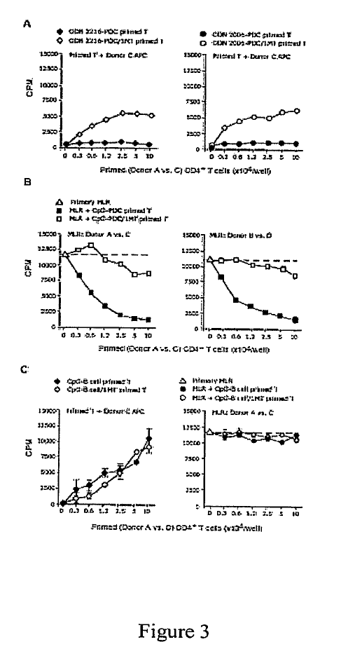

Addition of 1MT to the priming MLRs prevented CD4+ T cells from becoming anergic/hyporesponsive to subsequent alloantigen stimulation (FIG. 3A). On a per-cell basis, 1MT markedly reduced the development of Treg-mediated suppressor activity, measured as the ability of PDC-primed T cells to inhibit proliferation of autologous or allogeneic naive CD4+ T cells in MLR cultures (FIG. 3B). As a control for nonspecific effects of 1MT, we also tested 1MT in priming MLRs stimulated by allogeneic CPG ODN-activated B cells (which did not express significant amounts of IDO, see FIG. 2A). CD4+ T cells that were primed by B cells retained their alloreactivity upon re-stimulation (FIG. 3C, left panel), and did not acquire suppressive activity to inhibit the proliferation of naive CD4+ T cells in MLR cultures (FIG. 3C, right panel). In both of these controls using priming with CpG ODN-activated B cells, the addition of 1MT to the priming MLRs had no nonspecific effect on the subsequent T cell responses. Thus, the effect of 1MT was specifically to block the IDO-induced generation of Tregs by IDO-expressing PDCs.

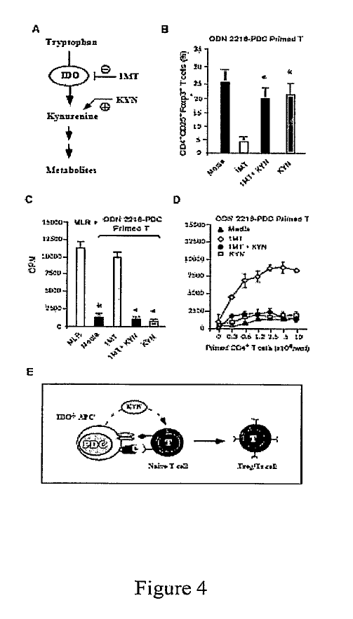

Downstream metabolites generated by IDO are critical for Treg induction. IDO degrades the essential amino acid Tryp to KYN, which is then metabolized by other enzymes to subsequent metabolites along the KYN pathway (Stone et al., Nat Rev Drug Discov 1:609-620). This example explores the mechanistic role of KYN pathway metabolites in the generation of Tregs by adding exogenous KYN to priming MLRs and bypassing the effect of 1MT and restoring Treg generation (diagrammed in FIG. 4A). This example demonstrates that exogenous KYN bypassed the blocking effect of 1MT, and restored CD4+CD25+Foxp3+ Treg generation in priming MLRs containing 1MT (FIG. 4B). KYN also restored the generation of functional suppressor activity (FIG. 4C), and hyporesponsiveness of primed T cells to alloantigen stimulation in MLR cultures (FIG. 4D).

These findings thus demonstrate that the effect of IDO on Treg generation can be reproduced by exogenous KYN when endogenous IDO is blocked, and implicate KYN pathway metabolites as the mechanism of IDO-induced Treg generation (FIG. 4E). This example provides the first evidence that human PDCs express IDO, and shows that PDCs employ the IDO pathway to induce the differentiation of CD4+CD25+Foxp3+ Tregs. This example implicate IDO-mediated production of metabolites in the KYN pathway as the mechanism of Treg generation by PDCs. This example adds to the growing number of studies indicating that APCs that have or acquire IDO expression are immunosuppressive (Mellor and Munn, Nat Rev Immunol 2004; 4:762-774; Munn et al., J Clin Invest 2004; 114:280-290; Fallarino et al. Int Immunol 2002; 14:65-68; Hwu et al., J Immunol 2000; 164:3596-3599; Mellor et al., J Immunol 2005; 175:5601-5605; and Munn et al., J Immunol 2004; 172:4100-4110). Several studies have shown that downstream Tryp metabolites such as KYN appear to have a direct suppressive effect on T cell responses, causing inhibition of proliferation and apoptosis of T cells (Frumento et al., J Exp Med 2002; 196:459-468; Terness et al., J Exp Med 2002; 196:447-457; and Fallarino et al., Nat Immunol 2003; 4:1206-1212). This example shows that exposure of naïve CD4+ T-cells to IDO-expressing PDCs induces the differentiation of Treg cells from naive CD4+ T cells. Blocking IDO with 1MT abrogates the generation of Tregs, but Treg generation is restored by adding exogenous KYN in the presence of 1MT. These data thus strongly implicate metabolites in the KYN pathway as participating in the differentiation of Tregs. Exogenous KYN can be spontaneously taken up by cells (Moffett et al., Exp Neurol 1997; 144:287-301) and metabolized to a variety of compounds along the KYN pathway, depending on the pattern of enzymes expressed by the cells (Werner-Felmayer et al., Biochim Biophys Acta 1989; 1012:140-147). Several of these metabolites have been shown to have an effect on T cells in vitro (Frumento et al., J Exp Med 2002; 196:459-468; Terness et al., J Exp Med 2002; 196:447-457; and Fallarino et al., Nat Immunol 2003; 4:1206-1212). Recently, the metabolite 3-hydroxyanthranilic acid and a synthetic structural analog N-(3,4-Dimethoxycinnamoyl) anthranilic acid (3,4-DAA), have been shown to inhibit inflammatory cytokine production by auto-reactive T cells, and reverse paralysis in a mouse model of experimental autoimmune encephalomyelitis (Platten et al., Science 2005; 310:850-855). The findings of this example provide evidence that the natural metabolites produced by the IDO/KYN pathway are involved in the de novo generation of human CD4+CD25+Foxp3+ Tregs by PDCs. These data suggest novel strategies for the use of PDCs as a means to induce CD4+ Tregs for tolerance induction, which may metabolites produced by the IDO/KYN pathway are involved in the de novo generation of human CD4+CD25+Foxp3+ Tregs by PDCs. These data suggest novel strategies for the use of PDCs as a means to induce CD4+ Tregs for tolerance induction, which may offer new opportunities in autoimmunity and transplantation (Taylor et al., Blood 2002; 99:3493-3499; Bushell et al., J Immunol 2005; 174:3290-3297; Tang et al., J Exp Med 2004; 199:1455-1465; Bluestone, Nat Rev Immunol 2005; 5:343-349). From a clinical standpoint, it may be even more relevant that exogenous KYN is able to reproduce the generation of Tregs even in the absence of endogenous IDO activity.

### Example 2

### Indoleamine 2,3-Dioxygenase Rapidly Activates Suppressor Functions of Regulatory T Cells

Indoleamine 2,3 dioxygenase (IDO) activity mediates T cell suppressive effects in inflammatory conditions associated with a diverse range of clinical syndromes. When induced to express IDO specific subsets of dendritic cells acquire potent T cell suppressive functions. This example shows that induced IDO activity also stimulates CD4+CD25+ regulatory T cells (Tregs) to acquire increased T cell suppressor functions. After treating mice with TLR9 ligands to induce IDO purified splenic Tregs rapidly acquired potent suppressor functions that blocked allospecific T cell responses elicited in vitro and in vivo. Genetic or pharmacologic ablation of IDO prevented stimulation of Treg suppressor functions. Moreover, Tregs selectively expressed the GCN2-dependent inducible stress response protein CHOP following IDO induction, and this response was also IDO-dependent. These findings indicate the hypothesis that IDO rapidly stimulates peripheral Tregs to acquire potent suppressive functions via activation of the GCN2-kinase mediated stress response to amino acid withdrawal.

### Material and Methods

Mice. All mice were bred in a specific pathogen-free facility. BM3 TCR transgenic mice IDO-deficient (IDO-KO) and GCN2-deficient (GCN2-KO) mice were described previously (Mellor et al. J Immunol 2005; 175:5601-5605; Munn et al. Immunity 2005; 22:1-10). All procedures involving mice were approved by the Institutional Animal Care and Use Committee.

CpG Oligonucleotides. CpG-ODNs (CpG no. 1826, TCCATGACGTTCCTGACGTT; (SEQ ID NO:4) and sequence matched non-CpG-B no. 2138, TCCATGAGCTTCCTGAGCTT (SEQ ID NO:5)) with fully phosphorothioate backbones were purchased from Coley Pharmaceuticals. Mice were injected with relatively high doses of ODNs (50 μg/mouse, i/v) as described (Mellor et al. J Immunol 2005; 175:5601-5605).

1-methyl-[D]-tryptophan (1mT). 1mT (catalog number 45, 248-3, Sigma) was prepared as a 20 mM stock solution in 0.1 N NaOH, adjusted to pH 7.4, and stored at −20° C. protected from light. For in vitro use, 1 mT was added to MLRs to a final concentration of 100 μM. For in vivo treatment, slow-release polymer pellets (*5 mg/day) containing 1 mT or vehicle alone were inserted under the dorsal skin as described (Munn et al. Science 1998; 281:1191-1193) 24 hours before CpG treatment.

Preparative flow cytometry to sort CD4+ T cells subsets. CD4+ T cell subsets were purified using a Mo-Flo cytometer as described (Mellor et al. J Immunol 2005; 175:5601-5605; Munn et al. Immunity 2005; 22:1-10).

Analytical flow cytometry. Intracellular CHOP staining was performed as described (Munn et al. (2005) Immunity 22:1-10), using antibody sc-7351 (Santa Cruz Biotechnology, Santa Cruz, Calif.).

Ex vivo T cell suppression assays. Suppression assays were performed by adding sorted CD4+ cells to T cell proliferation assays (72 hour thymidine incorporation assays) containing responder H-2K^{b}-specific splenocytes from BM3 TCR transgenic mice (nylon-wool enriched) and CD11c+ splenocytes (AutoMacs enriched) from CBK (H-2K^{b} transgenic CBA) mice prepared as described (Mellor et al. J Immunol 2005; 175:5601-5605).

Co-Adoptive Transfer. Purified (Mo-Flo sorted) CD4+CD25+ Tregs were prepared from spleens of CBK donor mice treated 24 hours previously with either 50 μg CpG or non-CpG. Tregs (1×10⁶/recipient) were mixed with nylon-wool enriched BM3 responder T cells (5×10⁶/recipient) and co-injected into CBK recipients (2-3 mice/group). Positive controls were CBK mice receiving BM3 T cells without Tregs; negative controls were CBA mice (lacking target antigen) receiving BM3 T cells. After 96 hours, mice were sacrificed and spleen cells stained with antibodies against CD8 (PerCP), CD25 (APC), H2K^{b} (PE) and biotinylated Ti98 (BM3 anti-clonotypic antibody, visualized with streptavidin APC (Tarazona et al. (1996) International Immunology 8:351-358). Except for Ti98, all antibodies were from Pharmingen (San Diego, Calif.).

### Results and Discussion

TLR9 ligands rapidly enhance Treg suppressor functions. To test if IDO activity enhanced Treg suppressor functions, mice were treated with relatively high systemic doses of TLR9 ligands (CpG), which induces splenic pDCs expressing CD19 (CD19+ pDCs) to express functional IDO. Following CTLA4-Ig or TLR9 ligand treatment, CD19+ pDCs acquired potent and dominant T cell suppressive functions that blocked CD8+ T cell responses elicited in vitro and in vivo (Mellor et al. Int Immunol 2004; 16:1391-1401; Baban et al. Int Immunol 2005; 17:909-919; Mellor et al. J Immunol 2005; 175:5601-5605; Mellor et al. J Immunol 2003; 171:1652-1655). Closely related CD19+ pDCs with IDO-dependent T cell suppressive properties also accumulated in lymphoid tissues draining sites of B16 melanoma tumor growth in mice (Munn et al. J Clin Invest 2004; 114:280-290).

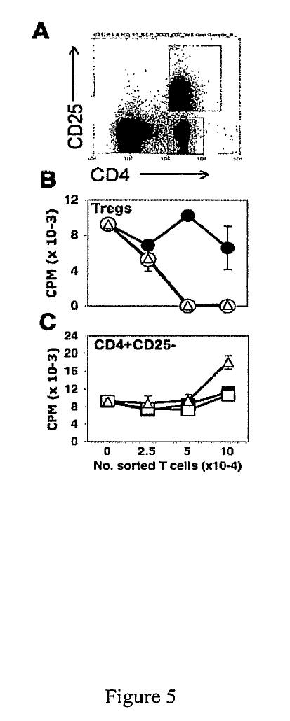

Purified Tregs from mice treated for 24 hours with TLR9 ligands (CpG #1826) suppressed proliferation of BM3 (H-2K^{b}-specific) CD8+ T cells when ≧5×10³ sorted Tregs were added to cultures containing BM3 T cells and APCs expressing H-2K^{b} (FIGS. 5A and 5B). Addition of IDO inhibitor, 1-methyl-[D]-tryptophan (1mT) to parallel cultures did not reverse suppression, indicating that IDO activity during culture was not essential for Treg-mediated suppression. Maximal Treg suppressor activity was also detected 18 hours after treating mice with CpG, but no increase in Treg suppressor functions was detected 12 hours after CpG treatment. In contrast, splenic Tregs from mice treated for 24 hours with sequence matched ODNs containing no CpG motifs (non-CpG, #2318) did not suppress BM3 proliferation, even when 104 purified Tregs were added to cultures. As expected, purified CD4+CD25− T cells from CpG or non-CpG treated mice had no significant effect on BM3 T cell proliferation (FIG. 5C). These data revealed that Treg suppressor functions increased rapidly following TLR9 ligation in vivo, and that IDO activity was not required for Treg mediated suppression measured ex vivo.

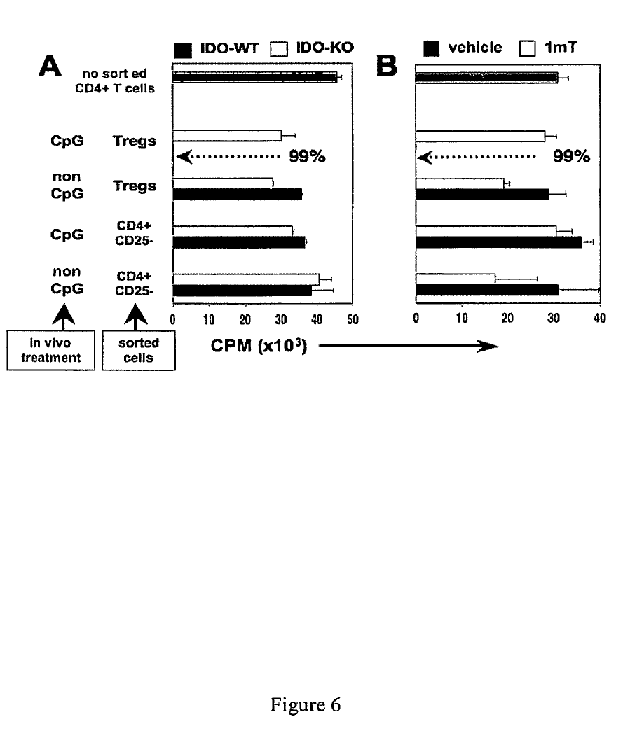

TLR9 ligands stimulate Treg suppressor activity by inducing functional IDO expression. To test if the stimulatory effects of in vivo CpG treatment on Treg suppressive functions were IDO-dependent, CpG was administered to IDO-deficient (IDO-KO) or wild type (IDO-WT) mice and tested if Tregs acquired increased suppressive functions. Purified Tregs isolated from IDO-KO mice exposed to CpG or non-CpG exhibited no significant increase in suppressor functions (FIG. 6A, white bars). As before, purified Tregs from IDO-WT mice acquired potent suppressor activity following CpG treatment (FIG. 6A, black bars), and purified CD4+CD25− T cells from IDO-KO or IDO-WT mice exposed to CpG or non-CpG had no significant effect on BM3 T cell proliferation.

An alternative approach was used to determine that IDO was essential for TLR9-mediated stimulation of Treg suppressive functions by treating IDO-WT mice with the pharmacologic IDO-inhibitor, 1-methyl-(D)-tryptophan (1mT) 24 hours before exposing them to CpG. As shown in FIG. 5B, treating IDO-WT Treg donor mice with IDO inhibitor prevented CpG-mediated stimulation of Treg suppressive functions (FIG. 6B, white bars). However, exposing mice to drug delivery vehicle alone prior to CpG treatment had no effect on CpG-mediated stimulation of Treg suppressor functions (FIG. 6B, black bars). Thus, an intact IDO gene and functional IDO enzyme activity were essential to stimulate increased Treg suppressor functions following TLR9 ligation. These data revealed that TLR9 ligands stimulated Treg suppressor functions indirectly by inducing functional IDO expression in vivo.

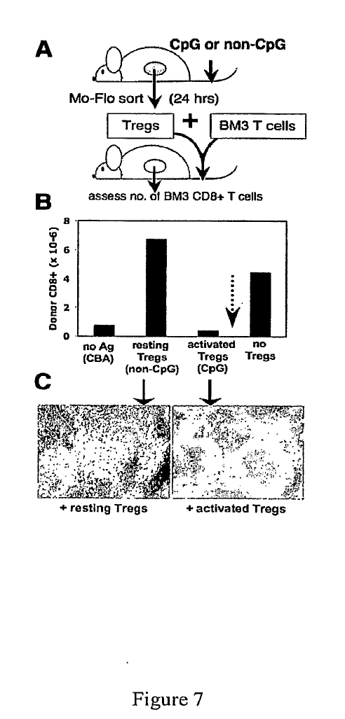

IDO-activated Tregs suppress alloreactive T cell responses elicited in vivo. Next, whether IDO-activated Tregs suppressed tissue destruction mediated by alloreactive T cells was assessed by co-injecting purified Tregs and splenocytes from BM3 TCR transgenic mice into recipient mice expressing H-2K^{b} alloantigen (FIG. 7A). In this model, BM3 CD8+ T cells undergo rapid clonal expansion and differentiate into cytolytic effectors that cause extensive tissue pathology and loss of tissue integrity in spleen, accompanied by significant reduction in spleen size and cellularity (Mellor et al. J Immunol 2003; 171:1652-1655; Tarazona et al. International Immunology 1996; 8:351-358). Co-adoptive transfer of BM3 T cells and Tregs from donor mice treated with non-CpG (resting Tregs) did not inhibit subsequent clonal expansion of BM3 T cells, relative to recipient mice that received BM3 T cells only (FIG. 7B). Moreover, resting Tregs did not prevent extensive tissue pathology accompanied by extensive infiltration of CD8α+ cells throughout remaining spleen tissues (FIG. 7C), consistent with clonal expansion and differentiation of cytolytic CD8+ T cells in these mice. In contrast, co-transfer of BM3 T cells and Tregs from donor mice treated with CpG (activated Tregs) prevented BM3 clonal expansion and spleen integrity was normal (FIGS. 7B and 7C). These outcomes revealed that Tregs from CpG-treated mice suppressed alloreactive T cell responses capable of causing extensive tissue pathology.

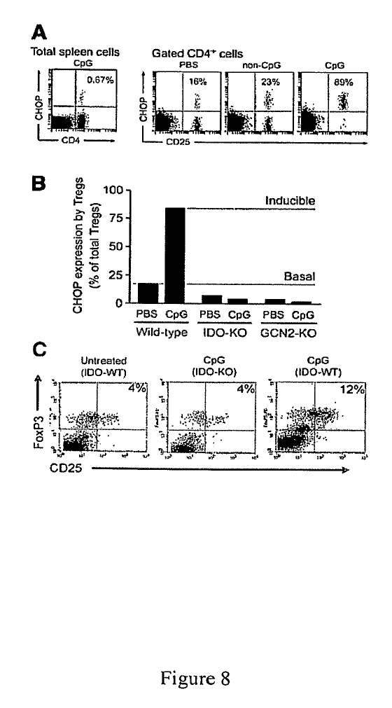

Tregs respond selectively to induced IDO by undergoing the GCN2-dependent stress response. It has recently been reported that IDO activated the GCN2-kinase dependent integrated stress response in naïve effector T cells blocking clonal expansion and differentiation in response to antigenic stimulation, which lead to T cell apoptosis and anergy (Munn et al. Immunity 2005; 22:1-10). As IDO also stimulates suppressive functions in Tregs, if IDO activated GCN2-kinase in Tregs was addressed by assessing CHOP expression, a downstream inducible gene controlled by GCN2-kinase (Munn et al. Immunity 2005; 22:1-10; Dong et al. Mol Cell 2000; 6:269-279; Harding et al. Mol Cell 2003; 11:619-633; Wek et al. (Biochem Soc Trans 2006; 34:7-11). Following CpG treatment, <1% of total splenocytes expressed CHOP, and all CHOP+ cells expressed CD4 (FIG. 8A). Three-color flow cytometric analyses of gated CD4+ cells showed that CHOP+ cells were confined to the CD4+CD25+ Treg population. In untreated mice a minor fraction of splenic Tregs (*15-20%) expressed CHOP. After CpG treatment, the majority of Tregs (80-90%) expressed CHOP, but CHOP expression was still restricted to Tregs. Treatment with non-CpG only slightly increased the number of CHOP+ Tregs over basal levels. However, CpG-induced CHOP expression was not observed in Tregs from IDO-KO mice (FIG. 8B), suggesting that Tregs in IDO-KO mice failed to undergo the GCN2-dependent stress response following TLR9 ligation. As expected, CpG also failed to induce CHOP in Tregs from GCN2-deficient (GCN2-KO) mice (Munn et al. Immunity 2005; 22:1-10). Thus, Tregs responded selectively to IDO by activating the GCN2-kinase dependent integrated stress response. As the transcription factor FoxP3 is essential for Treg suppressor functions (Fontenot et al. Nat Immunol 2003; 4:330-336), FoxP3 expression was evaluated in Tregs following CpG treatment (FIG. 8C). CpG treatment lead to a 3-4 fold increase in the number of FoxP3+ Tregs in IDO-WT mice (from 4-5% to 12-16%), but FoxP3 expression levels increased only marginally. In contrast, CpG treatment did not induce a significant increase in the number of FoxP3+ Tregs in IDO-KO mice, indicating that this response to TLR9 ligation was IDO-dependent. These outcomes support the hypothesis that induced IDO activity selectively affects Tregs, which respond by undergoing the GCN2-kinase dependent stress response to amino acid withdrawal.

This example shows that IDO activity stimulated rapid increase of Treg suppressor functions and activated the GCN2 stress response selectively in Tregs. Following IDO induction, Tregs suppressed robust alloreactive T cell responses elicited ex vivo and in vivo under conditions where Tregs from mice treated with control reagents (non-CpG) exhibited no detectable suppressor activity. These findings provide a potential explanation for the potent IDO-dependent suppressive effects of CD19+ pDCs, which constitute less than 10% of total splenic DCs (Baban et al. Int Immunol 2005; 17:909-919; Mellor et al. J Immunol 2005; 175:5601-5605). Hence, as well as direct suppression of effector T cell responses, CD19+ pDCs expressing IDO may also activate the suppressive functions of quiescent Tregs to promote bystander suppression. However, an alternative possibility is that TLR9 ligands acted directly to induce IDO expression in Tregs, as Tregs express TLRs (Wang et al. Semin Immunol 2006; 18:136-142), and T cells can be induced to express IDO in some circumstances (Curreli et al. Journal of Interferon and Cytokine Research 2001; 21:431-437; Boasso et al. Blood 2005; 105:1574-1581). Though we cannot exclude this possibility completely, quantitative RT-PCR analyses of RNA samples from purified Tregs revealed that CpG treatment did not induce IDO transcription in Tregs, suggesting that Tregs themselves were not the source of IDO activity that triggered increased suppressor functions.

The observations that CpG treatment induced selective CHOP expression in almost all splenic Tregs, and that this response did not occur in Tregs from IDO-KO mice, suggest that IDO-mediated tryptophan catabolism caused selective activation of the GCN2-dependent stress response to amino acid withdrawal in Tregs. The selectivity of this response was particularly striking because the GCN2-dependent stress response is a generalized response to amino acid withdrawal exhibited by all cell types (Wek et al. Biochem Soc Trans 2006; 34:7-11). Thus, additional signals may control the selective response of Tregs to IDO induction in the splenic microenvironment. Tregs might also require simultaneous TCR signals via recognition of constitutively expressed self-antigens on splenic APCs in order to activate suppressor functions (Hsieh et al. Immunity 2004; 21:267-277). It is unclear if an intact GCN2-kinase stress response is required for Tregs to acquire increased suppressor functions following IDO induction. Although GCN2-KO mice possess peripheral Tregs, the proportion of Tregs within the CD4+ T cell compartments is substantially reduced relative to wild-type mice (˜10 fold less), suggesting that Treg development and survival may be impaired in GCN2-KO mice.

Freshly isolated Tregs possess relatively weak suppressor functions, which increase significantly following mitogenic and antigenic activation (Thornton et al. Eur J Immunol 2004; 34:366-376; Nishikawa et al. J Exp Med 2005; 201:681-686; Yu et al. J Immunol 2005; 174:6772-6780). However, increased Treg suppressor activity takes some time to manifest, probably due to requirements for Treg proliferation and/or differentiation after TCR ligation. Moreover, Treg suppressor activity is antagonized by signals from activated DCs and TLR8 (Pasare and Medzhitov Science 2003; 299:1033-1036; Peng et al. Science 2005; 309:1380-1384). This example detected increased Treg suppressor activity as soon as 18 hours after mice were treated with TLR9 ligands. This suggests that Treg proliferation was not required for enhanced suppressor activity in our experimental system, and that the well documented immunostimulatory effects of TLR9 ligation were subordinate to the enhanced suppressive functions acquired by Tregs following IDO induction. It has been suggested that Tregs expressing surface CTLA4 might suppress T cell responses by inducing IDO via ligation of B7 (CD80/86) molecules expressed by DCs (Mellor et al. Int Immunol 2004; 16:1391-1401, Finger and Bluestone Nat Immunol 2002; 3:1056-1057; Fallarino et al. Nat Immunol 2003; 4:1206-1212). In the present example, IDO inhibitor did not block Treg suppression measured ex vivo, indicating that IDO was not mechanistically required for Treg-mediated suppression following IDO-dependent stimulation in vivo.

In summary, this example demonstrates that IDO triggers a rapid increase in suppressor functions of splenic Tregs. Clearly, IDO is not the only mechanism capable of activating Treg suppressor functions, especially as IDO-KO and GCN2-KO mice do not succumb to the lethal phenotype of Treg-deficient mice. However, the significance of the present study is that it identifies a novel checkpoint at which the Treg system can be regulated. This example also provides a mechanistic explanation for potent bystander suppression created by minor cohorts of IDO+ pDCs (Munn et al. J Clin Invest 2004; 114:280-290; Mellor et al. J Immunol 2003; 171:1652-1655). Thus, this example study identifies a mechanism that amplifies the direct suppressive effects of IDO+ pDCs by stimulating the suppressor functions of Tregs.

### Example 3

### Dendritic Cells from Tumor-Draining Lymph Nodes Directly Activate Mature Regulatory T Cells Via Indoleamine 2,3-dioxygenase

A subset of dendritic cells (DCs) in tumor-draining lymph nodes can express the immunoregulatory enzyme indoleamine 2,3-dioxygenase (IDO). This example shows that IDO expression by these DCs directly activates potent suppressor activity in regulatory T cells (Tregs). This IDO-induced form of activation affected only mature, CD4⁺CD25⁺Foxp3⁺ Tregs, and did not cause differentiation of new Tregs from precursor cells. IDO induced freshly-isolated, resting Tregs to become potently suppressive for bystander cells without the need for exogenous mitogen or in vitro pre-activation. IDO-induced activation showed a strict requirement for interaction of Tregs with MHC molecules on the IDO⁺ DCs, required an intact GCN2 kinase pathway in the Tregs, and caused Treg-mediated target cell suppression in a non-contact-dependent fashion requiring interleukin-10 and TGFβ. This example indicates that IDO-induced Treg activation allows the local immunosuppressive effects of IDO⁺ DCs in tumor-draining lymph nodes to be amplified and extended to contribute to systemic tolerance.

### Materials and Methods

Mice and Reagents. Reagents were from Sigma unless otherwise noted. 1-methyl-D-tryptophan (catalog number 45, 248-3, Sigma) was prepared as described (Munn et al., Immunity 2005; 22:633-642). Details of the various transgenic and knockout mouse models are given below.

Isolation of tumor-draining lymph node DCs. Tumors were initiated using 1×10⁶ B78H1•GM-CSF cells (a sub-line of B16 melanoma transfected with GM-CSF (Huang et al., Science 1994; 264:961-965) implanted in thigh of either B6 mice or IDO-KO mice on the B6 background, as described (Munn et al., J. Clin. Invest. 2004; 114:280-290). Inguinal LNs were removed for cell sorting. IDO⁺ DCs were enriched using high-speed MoFlo cell-sorting for CD11c⁺B220⁺ cells as previously described (Munn et al., J. Clin. Invest. 2004; 114:280-290).

Bystander-suppression assays. All experiments were repeated 3-5 times unless otherwise indicated. OT-I cells were sorted as CD8⁺ spleen cells, gated on the CD11c^{NEG}B220^{NEG} fraction to exclude DCs. Sorted DCs from TDLN were mixed with 1×10⁵ responder cells at a 1:40 ratio in V-bottom culture wells (Nunc). Sorted CD4⁺CD25⁺ Tregs (typically 90% Foxp3⁺) were added at 5000 per well unless otherwise specified. Sorted CD4⁺ A1 cells (1×10⁵) and CD11c⁺ DCs (1:40 ratio) from normal CBA spleen were added as bystander cells. All assays received 100 nM OVA peptide SIINFEKL (SEQ ID NO:1) and 100 nM H-Y peptide REEALHQFRSGRKPI (SEQ ID NO:6) (Zelenika et al., J. Immunol. 1998; 161:1868-1874). Some wells received one or more of the following: 200 uM 1MT; rat anti-IL-10-receptor neutralizing antibody (Pharmingen, clone 1B1.3a); or chicken anti-TGF-β1,-β2,-β3 antibody (R&D Systems, MAB1835). For transwell assays, 96-Multiwell insert plates (1 uM pore size, BD Falcon) were used and the number of cells in all groups was doubled.

Feeder layer. Plasmacytoid DCs and Tregs have been reported to require survival factors to maintain viability and function in vitro. Therefore, as a feeder layer for these cells we added T cell-depleted spleen cells (1×10⁵ sorted CD4^{NEG}CD8^{NEG} cells) to all assays, similar to other culture systems (Thornton et al., Eur. J. Immunol. 2004; 34:366-376). This feeder layer was necessary for Treg function but it was entirely nonspecific, in that it could be derived from any host regardless of MHC haplotype (H2^{b} or H2^{k}) or strain background (B6, CBA, Balb/c or 129), and could be from GCN2-KO, IDO-KO or Foxp3-KO mice. The feeder layer could also be fully replaced by a cocktail of recombinant cytokines (IFNα+IL-10+TGFβ), chosen for their published ability to support survival of pDCs and Tregs. Thus, the function of the feeder layer was supportive only.

Readouts for T cell proliferation. For CFSE assays, sorted A1 and OT-I cells were labeled with CFSE dye as previously described (Munn et al., Immunity 2005; 22:633-642). After 72 hours, assays were stained for CD4 vs CD8 and CFSE fluorescence analyzed gated on CD4⁺ (A1) and CD8⁺ (OT-I) populations. Because the bystander assays were high-density and crowded (2×10⁵ TCR-transgenic T cells proliferating in 200 ul medium), they could not support more than 2-3 rounds of cell division without feeding or subculturing. However, this was sufficient to unambiguously determine which populations were dividing and which were arrested.

Thymidine-incorporation assays were more quantitative than CFSE for performing titrations and comparing multiple groups. However, thymidine incorporation could not distinguish whether one or both responder populations were proliferating; and, like CFSE assays, the proliferating cells tended to plateau at some maximum achievable value per well, regardless of whether one or both populations were proliferating. However, in cases where all thymidine incorporation was inhibited (which was the readout of interest) then this unambiguously revealed that suppression of both populations had occurred. Differences between groups (suppression vs. no suppression) were significant at P<0.01 by ANOVA, and are shown by arrows in the figures.

Anti-CD3 proliferation and preactivation assays. For anti-CD3-induced Treg activity, bystander-suppression assays were performed using higher numbers of Tregs (up to 1:1 ratio of Tregs to bystander cells, instead of 1:20) and with the addition of 0.1 ug/ml αCD3 antibody (Pharmingen, clone 145-2C11). For pre-activation studies, 2×10⁴ Tregs were cultured with 1×10⁵ T-depleted spleen cells plus 0.1 ug/ml αCD3 antibody and 200 U/ml IL-2 (R&D Systems) for 48 hours. Activated Tregs were fragile, so they were gently pipetted and transferred without washing into readout MLRs comprising 1×10⁵ sorted CD8⁺ BM3 T cells (TCR-transgenic, anti-H2K^{b}) plus 1×10⁵ irradiated B6 spleen cells. Recovered Treg number approximated the initial starting number, and data are presented in all cases as the nominal starting number of Tregs. BM3 T cells already have a high affinity for their cognate antigen, and validation studies showed that there was no further effect on the readout assay from the αCD3 used to pre-activate the Tregs.

Flow cytometry. Details of the staining protocols are given below. For CHOP and Foxp3 staining, assays were set up without the A1 bystander cells, so that the Tregs were the only CD4⁺ cells in the system and thus could be unambiguously followed throughout the assay.

Adoptive transfer and ex-vivo Treg assay. The adoptive transfer model has been previously described (Munn et al., Immunity 2005; 22:633-642; and Munn et al., J. Clin. Invest. 2004a; 114; 280-290). Briefly, DCs were sorted from TDLNs (total CD11c⁺ cells), pulsed with SIINFEKL (SEQ ID NO:1) peptide, and 5×10⁴ DCs injected into anteriomedial thigh. For studies measuring ex vivo Treg suppressor activity, the recipient mice were pre-loaded with 5×10⁶ sorted CD8⁺ OT-I cells. After four days, the inguinal LNs draining the site of DC injection were removed, and the CD4⁺CD25⁺ Tregs were isolated by FACS sorting. A titration of Tregs was added to readout assays, comprising CD4⁺ A1 cells, CBA DCs, feeder layer, and H-Y peptide, all as described above. For CFSE proliferation studies, CD8⁺ OT-I (wild-type or GCN2-KO background) were sorted, labeled with CFSE, and 5×10⁶ cells injected intravenously into wild-type or GCN2-KO recipients. OVA-pulsed DCs from TDLNs were injected as above, and the inguinal (draining) LNs harvested after four days. LN cells were analyzed by FACS for CD8 vs 1B11 vs CFSE.

Mouse models. All animal studies were approved by the institutional animal use committee of the Medical College of Georgia. TCR-transgenic OT-I mice (CD8⁺, recognizing the SIINFEKL (SEQ ID NO:1) peptide of chicken ovalbumin in the context of H2K^{b} (Hogquist et al., Cell 1994; 76:17-27) and CHOP-KO (B6.129S-Ddit3^{tm1Dron}/J (Zinszner et al., 1998; Genes Dev 12:982-995)), both on the B6 background, were purchased from Jackson Laboratories (Bar Harbor, Me.). GCN2-KO mice inbred on the B6 background have been previously described (Munn et al., Immunity 2005; 22:633-642). OT-I mice bred onto the GCN2-KO background have been previously described (Munn et al., Immunity 2005; 22:633-642), and for this study were re-bred onto a pure B6 background. A1 mice (CBA background, anti-HY peptide) (Zelenika et al., J. Immunol. 1998; 161:1868-1874), BM3 (CBA background, anti-H2K^{b} (Tarazona et al., Int. Immunol. 1996; 8:351-358)) and IDO-KO mice (B6 and CBA backgrounds (Baban et al., Int. Immunol. 2005; 17:909-919; and Mellor et al., J. Immunol. 2003; 171:1652-1655)) were as described. H2-M mutant mice inbred on the B6 background were as previously described (Martin et al., Cell 1996; 84:543-550).

FACS staining Antibodies were from BD-Pharmingen unless otherwise noted. Anti-mouse CD25-APC conjugate (clone PC61, cat. # 17-0251-81) was from eBioscience: This conjugate gave brighter signal and better separation of CD25⁺ cells than other conjugates from other suppliers. For intracellular staining of CHOP, live cells were first blocked for 10 minutes with mouse Fc Block (BD Pharmingen) in 10% fetal calf serum medium, stained with anti-CD4-FITC for 30 minutes on ice, washed with PBS, then fixed and permeablized for 20 minutes in 250 ul Cytoperm/Cytofix solution (BD Pharmingen) on ice. All subsequent staining and wash steps were in BD Permwash solution. Fixed cells were stained with 1:100 dilution of monoclonal anti-gadd153/CHOP (sc-7351, Santa Cruz Biotechnology), washed, and stained with secondary monoclonal rat anti-mouse-IgG1-PE (#550083, BD Biosciences). This secondary antibody was selected because it did not cross-react with surface immunoglobulin on mouse B cells. For CHOP staining, assays were set up without A1 bystander cells, so that Tregs were the only CD4⁺ cells, and thus could be unambiguously followed throughout the assay. For Foxp3 staining, anti-Foxp3-PE antibody (clone FJK-16s) was obtained from eBioscience and used per the manufacturer's protocol. For Foxp3 staining, assays omitted A1 bystander cells and Tregs were identified by CD4 expression, as for CHOP staining.

### Results

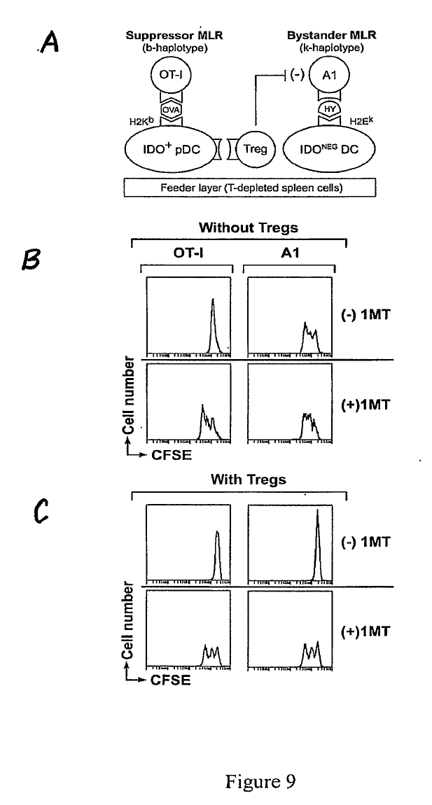

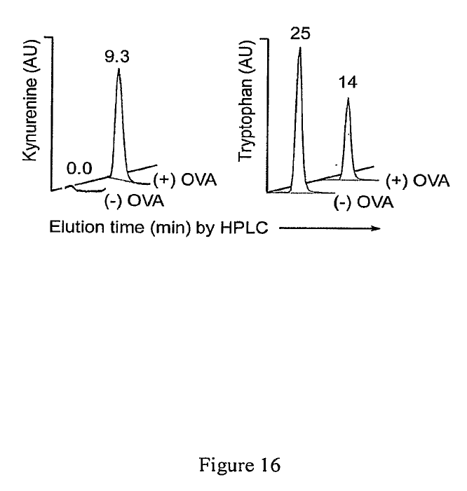

Activated Tregs create IDO-induced bystander suppression. Bystander suppression was measured using the system diagrammed in FIG. 9, comprising IDO⁺ DCs presenting antigen to OT-I T cells, plus a bystander population of IDO-negative DCs presenting antigen to A1 T cells. Because the different APCs were on different MHC backgrounds, the IDO⁺ DCs could not physically present antigen to the A1 T cells: Thus, any suppression of A1 must occur in a bystander fashion. IDO⁺ DCs were enriched from mouse TDLNs by FACS-sorting for the CD11c⁺ B220⁺ (plasmacytoid DC, pDC) fraction, as previously described (Munn et al., Immunity 2005; 22:633-642). This fraction included the specific subset of CD19⁺ CD11c⁺ B220⁺ cells that we have shown to comprise virtually all of the IDO-mediated suppression in TDLNs (Munn et al., J. Clin. Invest. 2004; 114:280-290). While CD19 is usually considered a marker for B cells, it is known that a subset of pDCs also expresses B-lineage markers (Corcoran et al., J. Immunol. 2003; 170:4926-4932; and Pelayo et al., Blood 2005; 105:4407-4415), and this same population of CD19⁺ DCs has been shown to mediate IDO-induced suppression in other settings (Baban et al., Int. Immunol. Immunol. 2005; 17:909-919; Mellor et al., J. Immunol. 2005; 175:5601-5605). As has been previously shown for human DCs (Munn et al., J. Immunol. 2004; 172:4100-4110), IDO⁺ DCs from TDLNs required triggering signals from T cells at the time of antigen presentation in order to express functional IDO enzyme activity; in the present system this signal was supplied by the activated OT-I cells. IDO activity was not triggered by the resting Tregs themselves, nor by OT1 cells without antigen, as shown in FIG. 16.

FIG. 9 shows an assay in which the OT-I and A1 cells were labeled with the fluorescent tracking dye CFSE. Each assay was performed in the presence or absence of Tregs, and with or without the IDO-inhibitor 1-methyl-D-tryptophan (1MT). The proliferation of OT-I cells was found to be governed strictly by the activity of IDO, irrespective of the presence of Tregs (i.e., OT-I was suppressed when IDO was active, and proliferated when IDO was blocked by 1MT). In contrast, suppression of bystander A1 cells depended on the presence of Tregs. Without Tregs (upper panels) A1 cells proliferated freely regardless of whether IDO was active or not. However, when Tregs were present (lower panels) proliferation of A1 became coupled to IDO, being suppressed when IDO was active. Thus, bystander suppression of A1 cells required both active IDO and the presence of Tregs. The Tregs themselves were not constitutively suppressive, since the groups receiving 1MT contained the same Tregs yet there was no suppression. Conversely, IDO itself did not appear to be directly suppressive to the bystander cells (e.g., via generation of soluble metabolites), because the A1 cells could proliferated freely in the presence of active IDO, as long as no Tregs were added. The activity of IDO in these cultures was confirmed by the fact that OT-I cells in the same wells were fully suppressed in a 1MT-reversible fashion, as shown in FIG. 9.

Comparison of IDO-induced activation vs. mitogen-induced activation of Tregs. In the literature, most reports have used one of two strategies to activate Tregs: occasionally, transgenic Tregs were are activated with a defined cognate antigen (Lerman et al., J. Immunol. 2004; 173:236-244); or, more often, polyclonal Tregs were activated with a mitogen such as anti-CD3 (Fontenot et al., Immunity 2005; 22:329-341; McHugh et al., Immunity 2002; 16:311-323; and Wan and Flavell, Proc. Natl. Acad. Sci. USA 2005; 102:5126-5131). The key observation from these reports is that activation is obligatory: in the absence of mitogen or cognate antigen, freshly-isolated Tregs do not display suppressor activity (Nishikawa et al., J. Exp. Med. 2005; 201:681-686; and Thornton et al., Eur. J. Immunol. 2004; 34:366-376). In contrast, in the present system IDO allowed freshly-isolated, resting Tregs to display spontaneous suppressor activity without exogenous mitogen. In order to directly compare the IDO-induced form of Treg activation with mitogen-induced activation, titrations of Tregs in bystander assays were performed (FIG. 9), in the presence or absence of 1MT to block IDO, and with or without addition of anti-CD3 mitogen. Since validation studies confirmed that the OT-I and A1 cells were already maximally stimulated by their respective cognate antigens and were not further stimulated by the addition of αCD3, the relevant effect of αCD3 was to activate the Tregs.

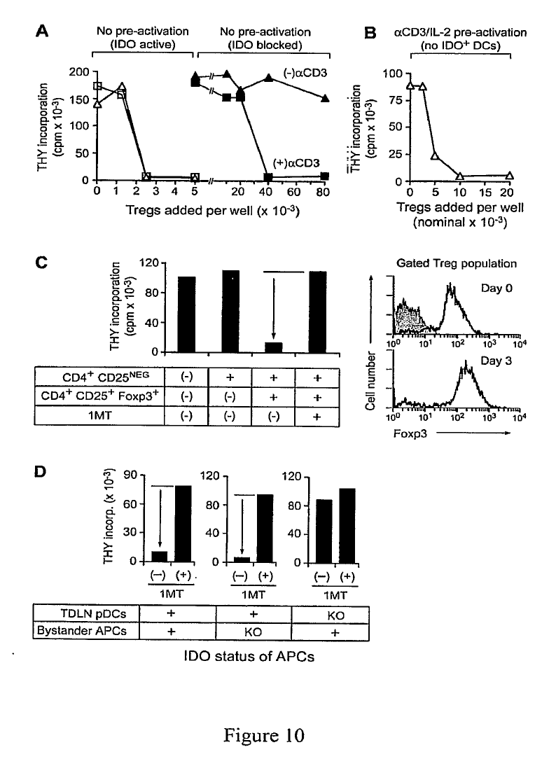

FIG. 10A shows that when IDO was active, less than 5000 Tregs were sufficient to completely suppress proliferation of 100,000 bystander cells. This suppression was equally effective with or without αCD3, indicating that IDO-activated Tregs had no further requirement for exogenous mitogen (and also indicating that suppression could not be overcome simply by providing a strong stimulus to the bystander cells, such as αCD3). In contrast, when IDO was blocked with 1MT, then even 50,000 Tregs showed no spontaneous suppressor activity (i.e., no suppression in the absence of αCD3 mitogen). The addition of αCD3 allowed Tregs to acquire suppressor activity when IDO was blocked (which was expected from the literature cited above). However, the form of Treg activity induced by αCD3 required 10-fold more Tregs, on a per-cell basis, than did IDO-induced Treg activity, in order to achieve comparable suppression. In these experiments, it was appropriate to quantitatively compare IDO-induced suppression and mitogen-induced suppression, because identical titrations were performed in identical replicate assays, using the same cell populations, differing only in the presence of 1MT and αCD3.

The quantitative level of αCD3-induced suppressor activity, although lower than that induced by IDO, was comparable to that reported in the literature for αCD3 and other mitogens (Fontenot et al., Immunity 2005; 22:329-341; and Wan and Flavell, Proc. Natl. Acad. Sci. USA 2005; 102:5126-5131). It has also been reported that the activity of resting Tregs can be increased by a period of in vitro pre-activation with αCD3 plus exogenous IL-2 (Thornton et al., Eur. J. Immunol. 2004; 34:366-376). To confirm that the starting Treg preparation was fully functional, FIG. 10B shows Tregs pre-activated in this fashion for two days, with suppressor activity then measured using a conventional allo-MLR readout (BM3 T cells stimulated by irradiated allogeneic splenocytes). Pre-activation with αCD3/IL-2 produced enhanced suppressor activity, comparable to that reported in the literature (Thornton et al., Eur. J. Immunol. 2004; 34:366-376), confinning that our Treg preparation was functional. Thus, the key finding from FIG. 10A was reinforced: that IDO was able to induced potent suppressor activity in freshly-isolated, resting Tregs, without the need for exogenous mitogen or in vitro pre-activation.

IDO acts directly on pre-existing Foxp3⁺ Tregs. FIG. 10C shows that IDO-induced bystander suppression required the presence of mature Tregs—i.e., it occurred only when sorted CD4⁺CD25⁺ Tregs were added to the system. These Tregs were typically >90% Foxp3⁺ by intracellular staining, as shown in the associated FACS histograms, and they remained Foxp3⁺ during IDO-induced activation. These cells thus represented lineage-committed, CD4⁺CD25⁺Foxp3⁺ Tregs. In contrast, the CD25^{NEG} (non-Treg) fraction of CD4⁺ T cells was not able to create bystander suppression when exposed to IDO (FIG. 10B). In other experiments, not shown, CD8⁺ T cells also failed to mediate bystander suppression. Thus, IDO acted directly on pre-existing, mature Foxp3⁺ Tregs, and did not cause de novo differentiation of new Tregs from CD25^{NEG} precursors.

In some situations, Tregs themselves have been reported to trigger expression of IDO in certain DCs (Fallarino et al., Nat. Immunol. 2003; 4:1206-1212). To test whether activated Tregs in our system might exert their effect by causing IDO upregulation in the bystander (CBA) DCs, assays were performed using bystander DCs derived from IDO-knockout (IDO-KO) mice. As shown in FIG. 10D, bystander suppression occurred equivalently whether the bystander DCs could express IDO or not. In contrast, if the TDLN DCs lacked IDO then bystander suppression was completely abrogated (FIG. 10C). Thus, IDO was required for activation of the Tregs, but the Tregs themselves did not function by inducing IDO in the bystander DCs.

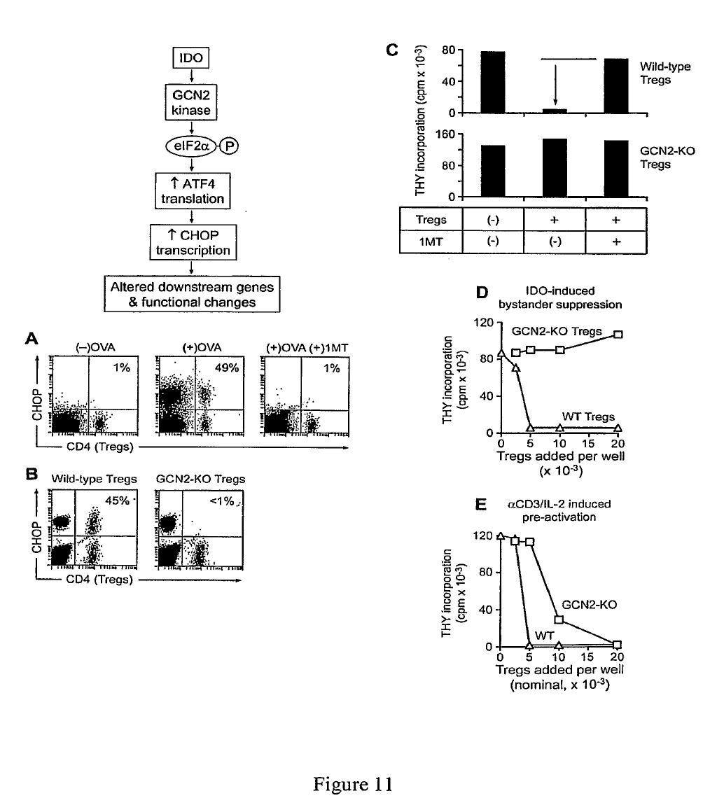

GCN2 kinase is required for IDO-induced activation of Tregs. As a molecular mechanism mediating the response to IDO, the GCN2 stress-kinase pathway was tested (FIG. 11). GCN2 is a kinase that responds to reduced levels of amino acids (Jousse et al., Biochem. Biophys. Res. Commun. 2004; 313:447-452), as might occur if IDO depleted the local supply of tryptophan. It has been previously shown that IDO activates GCN2-mediated signal transduction in CD8⁺ T cells, leading to cell-cycle arrest and anergy induction (Munn et al., Immunity 2005; 22:633-642). In the case of Tregs, it was hypothesized that GCN2 might trigger a downstream response pathway leading to enhanced suppressor activity. Activation of the GCN2 pathway can be detected by following the downstream marker gene CHOP (gadd153), as summarized schematically in FIG. 11. To determine whether IDO caused induction of CHOP in Tregs, assays were set up as in FIG. 9, but without the addition of bystander cells (since only the Tregs were of interest). Tregs were thus the only CD4⁺ cells in these cultures, and could be unambiguously identified on FACS analysis after activation. FIG. 11A shows that CHOP was not expressed in Tregs when IDO was not enzymatically active (i.e., if no antigen was presented to the OT-I cells). When cognate antigen was added and IDO thus became active (see FIG. 16) CHOP was induced in the OT-I cells, consistent with previous reports (Munn et al., Immunity 2005; 22:633-642). Under these conditions, CHOP was also induced in many of the Tregs (FIG. 11A, middle panel). CHOP upregulation was abrogated in both OT-I and Tregs by addition of 1MT, confirming that upregulation was IDO-dependent. FIG. 11B shows that CHOP expression was lost when Tregs were deficient in GCN2 kinase (GCN2-KO Tregs). In the same cultures, the OT-I cells still expressed CHOP normally indicating that IDO was active. Thus, IDO-induced CHOP expression in Tregs appeared to reflect activation of the GCN2 pathway in Tregs, as hypothesized.

Further consistent with the hypothesis, FIGS. 11C and 11D show that Tregs from GCN2-KO mice were unable to create IDO-induced suppression when tested in bystander assays. This inability to respond to IDO was not due to a global lack of function in GCN2-KO Tregs, since GCN2-KO Tregs that were pre-activated for 48 hrs with αCD3+IL-2 (using the system described in FIG. 10B) acquired suppressor activity that was approximately comparable to wild-type Tregs (FIG. 11E). Thus, GCN2-KO Tregs appeared to be profoundly but selectively deficient in their ability to respond to IDO-induced activation.

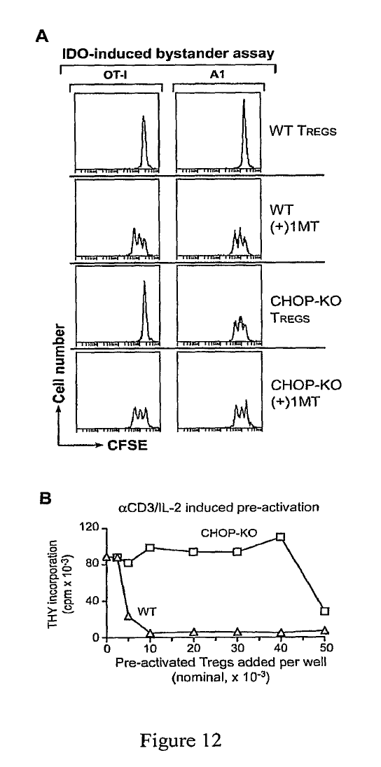

Bystander suppression is abrogated in CHOP-KO Tregs. To further test the hypothesis that IDO activated the GCN2 pathway in Tregs, a second point in the Integrated Stress Response (ISR) pathway downstream of GCN2 was targeted, to ask whether this produced a similar effect. For these studies, Tregs from mice deficient in the ISR-inducible transcription factor CHOP (Wek et al., Biochem. Soc. Trans. 2006; 34:7-11) were tested. FIG. 12A shows that CHOP-KO Tregs were unable to create IDO-induced bystander suppression, similar to the defect in GCN2-KO mice. Unlike GCN2-KO Tregs, however, CHOP-KO Tregs also displayed a significant quantitative defect in conventional Treg activity as well, shown by the reduced suppression following αCD3/IL-2 pre-activation (FIG. 12B). Thus, disrupting the CHOP gene, which was distal to GCN2 in the ISR pathway, abrogated the response to IDO, and also appeared to quantitatively affect mitogen-induced Treg activity as well.

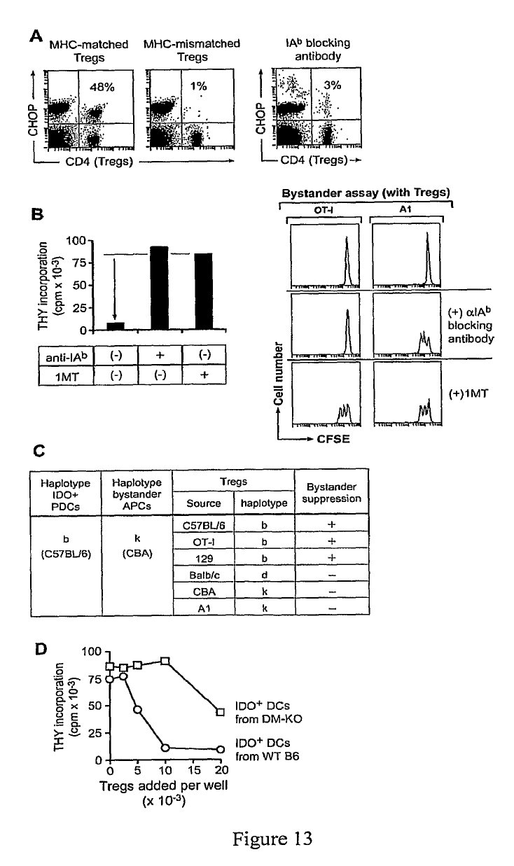

IDO-induced activation of Tregs requires contact with MHC. It has been previously shown that CHOP induction in CD8⁺ T cells requires two signals: one delivered via the GCN2 pathway, and the second via the T cell receptor (TCR) pathway (Munn et al., Immunity 2005; 22:633-642). Therefore it was asked whether Tregs required signaling via their TCR in order to become activated by the IDO/GCN2 pathway. In FIG. 13A, CHOP was again used as a read out for IDO-induced activation of GCN2. The TCR specificity of the polyclonal Tregs in our system was not known, but was assumed to be MHC-restricted, therefore it was asked whether CHOP induction required that the Tregs interact with MHC molecules expressed on the IDO⁺ DCs. FIG. 13A shows that when DCs and Tregs were MHC-matched, then CHOP was induced in the Tregs as expected (left-hand panel). However, if the Tregs and DCs were MHC-mismatched then CHOP was not induced (middle panel). In the same cultures CHOP was still induced in the OT-I cells, confirming that IDO was active. If the Tregs and DCs were MHC-matched, but physical interaction with the MHC molecules was interrupted using a blocking antibody against anti-IA^{b} (the MHC-II allele expressed by B6 mice), then CHOP induction was again abrogated (FIG. 13A, right-hand panel).

Tregs showed a similar strict requirement for interaction with MHC in order to create functional bystander suppression. FIG. 13B presents both thymidine-incorporation and CFSE readouts demonstrating that blocking MHC with anti-IA^{b} antibody abrogated IDO-induced bystander suppression. Here again, IDO was still active when IA^{b} was blocked, as shown by the IDO-dependent suppression of the OT-I cells in the same cultures (CFSE assays). FIG. 13C summarizes a series of bystander experiments using various combinations of haplotypes for the IDO⁺ DCs, bystander T cells and Tregs. These demonstrated that bystander suppression occurred only when the Tregs were MHC-matched to the IDO⁺ TDLN DCs, but not when they were matched to the IDO^{NEG} bystander DCs.

In theory, this MHC restriction might indicate only an interaction with the MHC framework elements, rather than actual antigen presentation. To test whether the peptide antigen presented by the MHC molecules also influenced Treg activation, H2-DM mutant mice were used. These mice have normal levels of cell-surface MHC-11 (Martin et al., Cell 1996; 84:543-550), but the large majority of these molecules contain only the Class-II Associate Invariant-chain Peptide (CLIP), rather than the normal repertoire of peptide antigens. Tumors were grown in H2-DM^{-/-} hosts, then H2-DM^{-/-} pDCs were isolated from TDLNs and used as the IDO-expressing DCs in bystander-suppression assays. Control assays received TDLN pDCs from wild-type B6 mice. In all assays, the Tregs were from the same wild-type B6 donors. FIG. 13D shows that the IDO⁺ DCs derived from H2-DM^{-/-} hosts were significantly impaired in their ability to activate Tregs for bystander suppression (requiring approximately four-fold more Tregs to achieve comparable suppression). Thus, within the constraints of the model, this suggested that the interaction of Tregs with IDO⁺ DCs included a significant contribution of the specific antigen presented by MHC-II. Experiments using CFSE labeling confirmed that the defect in H2-DM^{-/-} DCs lay in their ability to activate Tregs for bystander suppression, not in their direct IDO-mediated suppression of OT-I, which was intact.

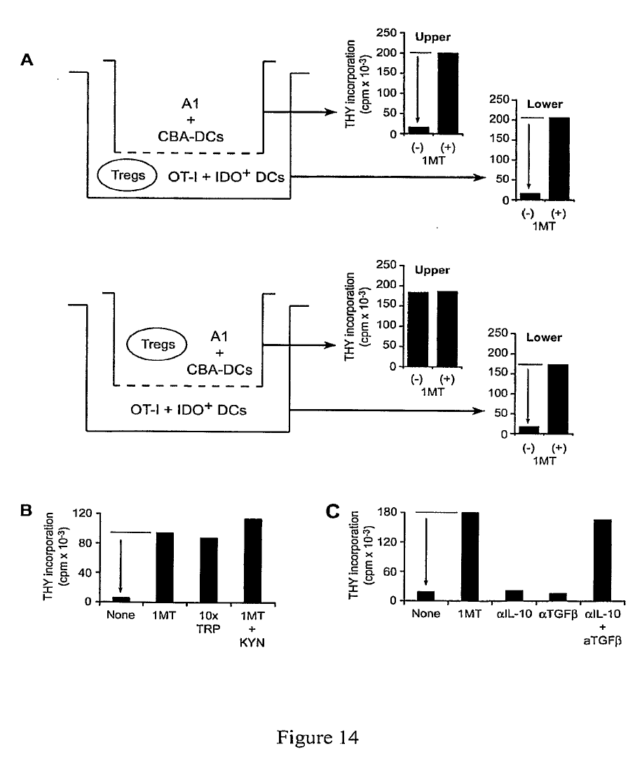

Suppression by IDO-activated Tregs does not require cell-cell contact. Next it was asked whether IDO-activated Tregs required physical contact with their target bystander cells in order to suppress them. The molecular mechanism of Treg-mediated suppression is still controversial (reviewed by Wing et al., Int. Immunol. 2006; 18:991-1000; and Bluestone and Tang, Curr. Opin. Immunol. 2005; 17:638-642) but most in vitro studies have found that conventional suppressor activity by Foxp3⁺ Tregs is contact-dependent. FIG. 14A shows results of bystander-suppression assays performed in transwell plates, in which the bystander cells (A1 T cells plus associated CBA DCs) were separated from the IDO⁺ DCs and OT1 cells by a microporous membrane. The Tregs were placed in the lower chamber, where they could be activated by the IDO⁺ DCs but could not physically contact the A1 bystander cells. At the end of the assay, both the upper and lower chambers were pulsed with tritiated thymidine, and T cell proliferation quantitated separately.

As shown in the top panel of FIG. 14A, Tregs in contact with the IDO⁺ DCs, but physically separated from the bystander cells, were still able to fully suppress the bystander cells. Suppression was triggered by IDO, as shown by the fact it was abrogated by 1MT. However, the suppression itself was mechanistically due to the activated Tregs, not to IDO, as shown by negative-control cultures in which the Tregs were shifted to the upper chamber (shown in the second panel of the figure). It is known from FIG. 13 that Tregs required physical contact with MHC molecules on the IDO⁺ DCs in order to be activated; therefore, moving them to the upper chamber prevented their activation by IDO. Under these conditions, suppression of A1 cells was completely lost. In these experiments, IDO itself remained active in the lower chamber, as shown by the 1MT-reversible suppression of OT-I cells in that chamber. Despite the presence of active IDO, the bystander cells were no longer suppressed in the absence of IDO-activated Tregs. Identical results were obtained when the Tregs were omitted entirely, but the ideal control was simply to prevent the IDO-induced form of activation by moving the Tregs to the upper chamber. Thus, IDO-activated Tregs were able to suppress bystander cells via a mechanism mediated by soluble factors, and which did not require cell-cell contact.

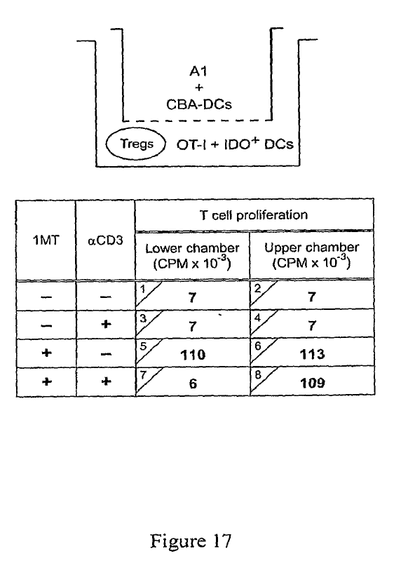

In contrast, conventional Treg activity (such as produced by αCD3) is reported to be contact-dependent (Wing et al., Int. Immunol. 2006; 18:991-1000). Therefore, whether one could discriminate αCD3-induced Treg activity in this system from IDO-induced Treg activity on the basis of contact dependence was addressed. Transwell experiments were performed as in FIG. 14A, but using 10-fold more Tregs and with the addition of αCD3 mitogen. These studies are shown in FIG. 17). The results clearly distinguished αCD3-induced suppression (which was dependent on αCD3, was not blocked by 1MT, and was strictly contact-dependent) from IDO-induced suppression (which was indifferent to αCD3, was blocked by 1MT, and was not contact-dependent).

Further consistent with the hypothesis that the soluble factor mediating bystander suppression was not derived from IDO itself, the addition of excess tryptophan to bystander assays abrogated suppression (FIG. 14B). If IDO were generating immunosuppressive metabolites from tryptophan, then adding excess tryptophan would not be predicted to reverse suppression (it should generate more metabolites); whereas excess tryptophan does overcome tryptophan depletion and GCN2-mediated responses to IDO (Munn et al., Immunity 2005; 22:633-642). Similarly, adding excess kynurenine to assays in which IDO was blocked with 1MT did not recapitulate suppression (FIG. 14B). These studies reinforced the more definitive data from the transwell system suggesting that activated Tregs, not IDO itself, were the proximate cause of bystander suppression.

Two specific soluble factors, IL-10 and TGFβ, have been implicated in certain forms of Treg-mediated suppression. Although not usually though to be involved in suppression by CD4⁺CD25⁺Foxp3⁺ (Wing et al., Int. Immunol. 2006; 18:991-1000), they are important in other types of regulatory T cell activity. FIG. 14C shows data from bystander-suppression assays in which antibodies were added to block the IL-10-receptor (IL-10R) and neutralize TGFβ. Blocking either the IL-10R or TGFβ pathway alone was not sufficient to reverse suppression, but blocking both together restored T cell proliferation. Thus, IL-10 and TGFβ were implicated as candidate soluble factors acting coordinately to contribute to bystander suppression created by IDO-activated Tregs.

IDO⁺ DCs activate Tregs in vivo. To test whether IDO could activate Tregs in vivo, CD11c⁺ DCs were isolated from TDLNs and adoptively transferred into new hosts without tumors. Recipient mice had been pre-loaded with a population of OT-I T cells, and the DCs were pulsed with SIINFEKL (SEQ ID NO:1) antigen prior to adoptive transfer. Four days later, the endogenous host Tregs were isolated from the lymph nodes draining the site of DC injection, and tested for spontaneous suppressor activity in a readout assay consisting of A1 T cells stimulated by normal CBA DCs and H-Y peptide. Thus, all of these cell populations were similar to the bystander assay shown in FIG. 9, except that the IDO-induced activation step had to occur in vivo.

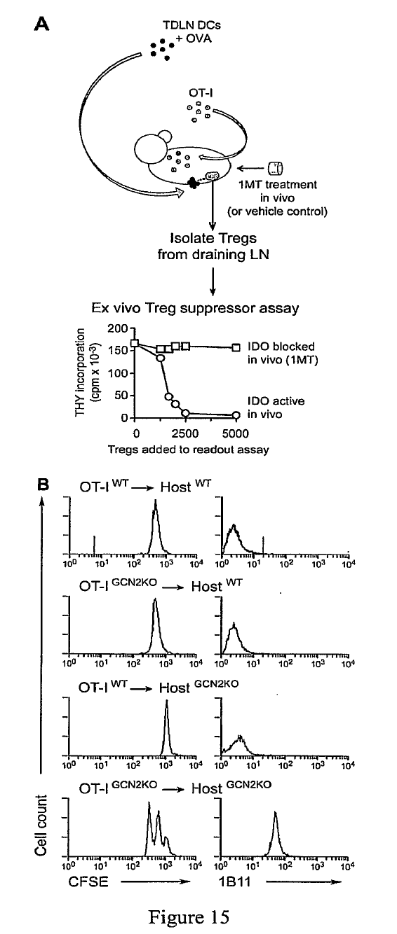

FIG. 15A shows that Tregs exposed to IDO⁺ DCs in vivo became activated for potent ex vivo suppression. This suppression was only induced if IDO was functionally active; when IDO activity was blocked by treating the recipient mice with 1MT, then the Tregs did not develop ex vivo suppressor activity. Since no mitogen was included in the readout assay, freshly-isolated Tregs would not be expected to display spontaneous suppressor activity unless they had been pre-activated in vivo. This had been shown above in FIG. 10A, and is consistent with the literature (Nishikawa et al., J. Exp. Med. 2005; 201:681-686; and Thornton et al., Eur. J. Immunol. 2004; 34:366-376. Thus, as predicted by our in vitro model, exposure of Tregs in vivo to IDO⁺ DCs resulted in activation of potent Treg suppressor activity.

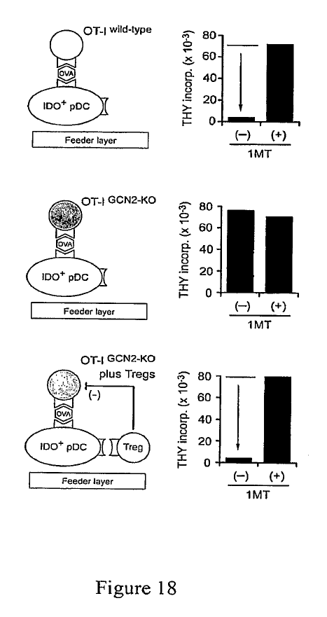

T cell inhibition by IDO⁺ DCs in vivo is mediated by both host and target cell GCN2. Finally, it was asked if there was evidence that IDO-activated Tregs were suppressive for T cells in vivo. To perform these studies, advantage was taken of the fact that GCN2-KO effector T cells (OT-I cells on the GCN2-KO background) were known to be refractory to direct suppression by IDO (Munn et al., Immunity 2005; 22:633-642). Although these cells were indifferent to IDO itself, it was found that they remained fully susceptible to Treg-mediated bystander suppression that was triggered by IDO (see FIG. 18). This allowed adoptive transfer studies, similar to FIG. 15A, to be performed in which GCN2 was knocked out either in the transferred OT1 cells, or in the recipient hosts mice (the source of the Tregs in vivo), or both. FIG. 15B shows an experiment using CFSE-labeled OT1 target cells (GCN2-KO or WT) pre-loaded into host mice (GCN2-KO or WT), and then challenged with antigen-pulsed IDO⁺ TDLN DCs. These studies showed that, as long as the host was GCN2-sufficient, even GCN2-KO OT-I cells were still unable to activate, despite the fact that they were indifferent to direct suppression by IDO. It was only when the host mice were also made GCN2-deficient that the OT-I^{GCN2-KO} cells became able to proliferate. Proliferating OT-I^{GCN2-KO} were able to upregulate the cytotoxic T cell activation marker 1B11 (Harrington et al., J. Exp. Med. 2000; 191:1241-1246), shown in the right-hand histograms, as a second marker of response to antigen. Taken together, these data were consistent with the existence of a population of IDO-inducible, GCN2-dependent suppressor cells in vivo; which is hypothesized to correspond to the IDO-activated Tregs isolated from the same lymph nodes, using the same model, in FIG. 15A.

As shown in FIG. 16, antigen presentation to OT-I cells is required to trigger functional IDO enzyme activity. In FIG. 16 functional IDO activity was measured as tryptophan depletion and kynurenine production in culture supernatants. Bystander-suppression assays were set up containing all of the cell populations described in FIG. 9, including Tregs. To increase the concentration of metabolites in the supernatants, each well contained 5 times the usual number of each cell type. Parallel assays were performed with and without the cognate OVA peptide (SIINFEKL (SEQ ID NO:1)) to activate the OT-I cells (both assays received the H-Y antigen for the A1 cells). Supernatants were harvested after 72 hours and analyzed by HPLC as described (Munn et al., J. Immunol. 2004; 172:4100-4110). The HPLC traces show the kynurenine and tryptophan peaks for the two treatment groups (with and without OVA). The concentration (in uM) of tryptophan and kynurenine in the medium is shown above each peak, interpolated from a standard curve. IDO only became enzymatically active (i.e, produced kynurenine and depleted tryptophan) when the pDCs presented antigen to OT-I.

As shown in FIG. 17, αCD3-induced Treg suppressor activity requires cell-cell contact, and is distinct from IDO-induced suppressor activity. Bystander-suppression assays were performed in transwell plates, with the bystander cells in the upper chamber (A1 T cells plus CBA DCs) and the IDO⁺ DCs, OT-I cells and Tregs in the lower chamber. Tregs were added at an increased ratio of 1:2 relative to the OT-I cells, instead of the usual 1:20. (Feeder cells were also in the lower chamber). Parallel assays received 1MT and/or αCD3, as shown in the table. Proliferation in each chamber was measured separately by thymidine incorporation, and each proliferation result is numbered in the table for ease of reference. Consistent with FIG. 14A, the IDO-induced form of Treg activity operated via a soluble, non-contact factor, as shown by the fact that A1 cells in the upper chamber were suppressed when IDO was active (seen by comparing group #2 vs. #6). In contrast, the αCD3-induced form of suppression (defined as the suppression occurring in the presence of 1MT but requiring αCD3) did not affect the A1 cells in the upper chamber (group #6 vs. #8). However, the Tregs were activated by αCD3, as shown by the fact that the OT-I cells in the lower chamber were suppressed when αCD3 was added (compare group #5 vs. #7). Thus, unlike the IDO-induced form of Treg activity, the αCD3-induced form was contact-dependent and could not affect cells in the upper chamber. This was consistent with previous published reports (Wing et al., Int. Immunol. 2006; 18:991-1000).

As shown in FIG. 18, OT-I cells that lack GCN2 are refractory to direct IDO-mediated suppression, but are sensitive to Treg-mediated suppression. TDLN pDCs were used to present SIINFEKL peptide to OT-I cells, which were either wild-type OT-I or OT-I^{GCN2-KO} (OT-I bred onto the GCN2-KO background). Wild-type OT-I were suppressed by IDO (top panel), but OT-I^{GCN2-KO} cells were resistant to direct suppression by IDO (middle panel), as previously described (Munn et al., Immunity 2005; 22:633-642). In the bottom panel, Tregs were included in the assay along with the OT-I^{GCN2-KO} responders. Now, even though the OT-I^{GCN2-KO} were themselves refractory to the direct effects of IDO, they were suppressed by the IDO-activated Tregs. Thus, Treg-mediated suppression was distinct from direct IDO-mediated suppression, and did not require an intact GCN2 pathway.

### Discussion

The present example demonstrates that IDO⁺ DCs possess the ability to directly and rapidly activate the latent suppressor function of resting Tregs. This novel form of Treg activation was still TCR-driven (i.e., it was restricted on MHC expressed by the DCs), and it affected only mature, differentiated CD4⁺CD25⁺Foxp3⁺ (“natural”) Tregs. Thus, it resembled in some ways the conventional Treg activity reported in the literature (Wing et al., Int. Immunol. 2006; 18:991-1000). However, IDO-induced Treg activation did not require mitogens such as anti-CD3 in order to trigger suppressor activity, nor did it require a period of in vitro pre-activation in order to produce potent, antigen-independent suppression of target cells. When IDO was active, even a small number of freshly-isolated, resting Tregs was able to completely suppress a large population of target T cells, driven only by the MHC molecules naturally expressed on the IDO⁺ DCs, and whatever cognate antigen was presented in the context of this MHC.

This raises the question of whether the IDO-induced form of suppression was mechanistically distinct from αCD3-induced suppression, or merely represented a quantitative increase in the same suppressive mechanism. This is difficult to definitively answer at present, because the molecular mechanism of conventional Treg activity is still controversial (Bluestone and Tang Curr. Opin. Immunol. 2005; 17:638-642; and Wing et al., Int. Immunol. 2006; 18:991-1000). However, it is suspected that IDO-induced suppression represents a distinct molecular mechanism. This is suggested by the fact that cell-cell contact was required only for the initial, IDO-induced Treg activation step, but not for the suppression of target cells (see FIG. 14A). In contrast, most previous studies of CD4⁺CD25⁺Foxp3⁺ Tregs have reported a contact-dependent mechanism of suppression (Wing et al., Int. Immunol. 2006; 18:991-1000), and it was found that conventional αCD3-induced suppression to be contact-dependent in our system. Thus, the two suppressor mechanisms appear distinct. In addition, with was found that GCN2-KO Tregs had near-normal levels of conventional αCD3-induced suppression, yet completely lacked any detectable IDO-induced Treg activity. Together, these findings suggest that IDO-induced Treg activity constitutes a mechanistically distinct suppressor pathway, different from the conventional αCD3-induced pathway.

That said, CHOP-KO Tregs displayed a partial quantitative defect in conventional αCD3-induced suppression, in addition to their complete lack of IDO-induced suppression. Thus, it is possible that the two suppressor pathways may share common elements at some point, even though they appear mechanistically distinct by the above criteria. The CHOP transcription factor, which lies further down the multi-functional Integrated Stress Response (ISR) pathway than GCN2, may be involved in additional signaling pathways; consistent with this, it is known that CHOP-KO mice have a number of immunologic abnormalities (Endo et al., J. Immunol. 2006; 176:6245-6253). Overall, the role of the ISR pathway in T cell biology is not yet fully elucidated. However, it has been previously shown that IDO inhibits CD8⁺ T cell activation and creates antigen-specific anergy by activating the GCN2/ISR pathway (Munn et al., Immunity 2005; 22:633-642). Others have shown that resting CD4⁺ T cells from GCN2-deficient mice are refractory to IDO-induced differentiation of new Tregs in vitro (Fallarino et al., J. Immunol. 2006; 176:6752-6761). Recently, helper CD4+ cells undergoing Th1/Th2 differentiation in vivo also were found to show marked ISR activation, although the mechanism of this is not yet known (Scheu et al., Nat. Immunol. 2006; 7:644-651). Thus, the ISR is emerging as a previously unappreciated regulatory pathway in T cell biology, with different downstream effects depending on the type of T cells involved.

The novel IDO-induced form of Treg activation that we describe is likely to represent a specialized pathway relevant specifically to those contexts in which IDO is important, rather than a generalized pathway of Treg activation. Consistent with this, the knockout mice used in this study (IDO-KO, GCN2-KO and CHOP-KO) did not display the spontaneous autoimmune phenotype seen in mice with a global defect in Tregs (e.g., Foxp3-deficient mice). This selective phenotype was expected, because the loss of IDO itself does not cause spontaneous global autoimmunity. Rather, mice in which IDO is acutely blocked show highly selective defects: e.g., rejection of allogeneic pregnancies (Muller et al., Nat. Med. 2005; 11:312-319; and Munn et al., Science 1998; 281:1191-1193), loss of ability to be tolerized by agents such as CTLA4-Ig (Grohmann et al., Nat. Immunol. 2002; 3:985-1109; and Mellor et al., J. Immunol. 2003; 171:1652-1655), and rapid death from otherwise survivable autoimmune inflammation (Gurtner et al., Gastroenterology 2003; 125:1762-1773). More beneficially, blocking IDO allows tumor-bearing mice to mount immune-mediated rejection of established tumors following chemotherapy, rather than permitting the tumors to grow unchecked (Muller et al., (2005) Nat. Med. 11, 312-319). Thus, the biologic role for IDO appears to lie in certain specific forms of acquired peripheral tolerance, including tolerance to tumors.

To date, however, it has been unclear how an apparently localized mechanism such as IDO could create such powerful systemic effects. Now, by elucidating the link between IDO expression and activation of the potent Treg system, we provide one possible mechanistic explanation for the systemic effects of IDO. The pathway of Treg activation that we describe is different from the well-known ability of certain DCs to cause the differentiation of new Tregs from uncommitted progenitors (Jonuleit et al., Trends Immunol. 2001; 22:394-400). IDO may also contribute to this process of de novo Treg differentiation as well (Fallarino et al., J. Immunol. 2006; 176:6752-6761). However, all studies to date have consistently found that de novo differentiation of Tregs is slow, occurring over many days. Therefore, this could not be the mechanism of IDO-induced bystander suppression, which must occur rapidly (within hours) in order to suppress T cells prior to their first cell division. The present example shows that IDO-induced Treg activation affects only mature, fully-differentiated CD4⁺CD25⁺Foxp3⁺ Tregs, and has no effect on the uncommitted CD25^{NEG} population of CD4⁺ T cells.

The present example indicates that the biologic significance of IDO-induced Treg activation is that it allows the immunosuppressive effects of IDO to extend beyond those T cells to which the IDO⁺ DCs physically present antigen. Via the activation of Tregs, the immunoregulatory effects of IDO⁺ DCs can be amplified and extended to suppress neighboring T cells, and perhaps to create systemic tolerance as well. As recently discussed (Munn and Mellor The tumor-draining lymph node as an immune-privileged site. Immunol. Rev. 2006 (in press)), this could have profound implications for the many TDLNs that harbor an abnormally increased population of IDO⁺ DCs. The present findings suggest that this small population of IDO⁺ DCs may be able to functionally suppress the entire TDLN, converting it from a normally immunizing milieu into an immunosuppressive and tolerogenic microenvironment.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

For any method disclosed herein that includes discrete steps, the steps may be conducted in any feasible order. And, as appropriate, any combination of two or more steps may be conducted simultaneously.

1. The method of claim 5, wherein the inhibitor of IDO suppresses the induction of regulatory T cells (Tregs) in the subject.

2. The method of claim 5, wherein the inhibitor of IDO suppresses the generation or reactivation of regulatory T cells (Tregs) in the subject.

3. The method of claim 5, wherein the inhibitor of IDO is 1-methyl-tryptophan (1-MT).

4. The method of claim 3, where 1MT is selected from the group consisting of a D isomer of 1-MT, a L isomer of 1-MT, and a racemic mixture of 1-MT.

5. A method of reducing immune suppression mediated by regulatory T cells (Tregs) in a subject, the method comprising administering to the subject an inhibitor of indoleamine-2,3-dioxygenase (IDO) in an amount effective to enhance an immune response.

6. A method to reduce the induction of antigen-specific regulatory T cells in a subject, the method comprising administering to the subject an effective amount of such an antigen in combination with an inhibitor of IDO.

7. The method of claim 6 wherein the antigen is a tumor antigen.

8. The method of claim 6 wherein the antigen is a viral antigen.

9. A method to enhance the immune response in a subject to a vaccine antigen, the method comprising administering to the subject the vaccine antigen, a CpG oligonucleotide (ODN), and an inhibitor of indoleamine-2,3-dioxygenase (IDO).

10-12. (canceled)

13. The method of claim 14, wherein the metabolic breakdown product of tryptophan is selected from the group consisting of L-kynurenine, kynurenic acid, anthranilic acid, 3-hydroxyanthranilic acid, quinolinic acid, picolinic acid, and analogs thereof.

14. A method of generating regulatory T cells (Tregs) in a subject, the method comprising administering to the subject a metabolic breakdown product of tryptophan, or an analog of a metabolic breakdown product of tryptophan.

15-36. (canceled)

37. The method of claim 5, the method further comprising administering an agent selected from the group consisting of a CpG oligonucleotide, an inhibitor of a GCN2 kinase, a vaccine, and a chemotherapeutic agent.

38. (canceled)


SEQUENCE LISTING (6 sequences)

SEQ ID NO: 1 - protein, 8 aa, artificial
  OVA peptide
SIINFEKL

SEQ ID NO: 2 - DNA, 19 bp, artificial
  CpG oligonucleotide
ggggacgatcgtcgggggg

SEQ ID NO: 3 - DNA, 24 bp, artificial
  CpG oligonucleotide
tcgtcgttttgtcgttttgtcgtt

SEQ ID NO: 4 - DNA, 20 bp, artificial
  CpG oligonucleotide
tccatgacgttcctgacgtt

SEQ ID NO: 5 - DNA, 20 bp, artificial
  CpG oligonucleotide
tccatgagcttcctgagctt

SEQ ID NO: 6 - protein, 15 aa, artificial
  H-Y peptide
REEALHQFRSGRKPI